(19) United States
(12) Patent Application Publication
(10) Pub. No.: US 2026/0224191 A1
(43) Pub. Date: Aug. 6, 2026

(54) SYSTEMS AND METHODS FOR THE CHARACTERIZATION, PREDICTION, OR TREATMENT OF PROGRESSIVE EYE CONDITIONS

(71) Applicants: Cornell University, Ithaca, NY (US); Institut National de la Sante et de la Recherche Medicale (Inserm), Paris (FR)

(72) Inventors: Jonathan Mamou, New York, NY (US); Cameron Hoerig, New York, NY (US); Quan V. Hoang, Singapore (SG); Stefan Catheline, Lyon (FR); Johannes Aichele, Pfaffhausen (CH)

(73) Assignees: Cornell University, Ithaca, NY (US); Institut National de la Sante et de la Recherche Medicale (Inserm), Paris (FR)

(21) Appl. No.: 19/153,229

(22) PCT Filed: Jan. 31, 2024
(86) PCT No.: PCT/US2024/013825

§ 371 Date: Aug. 1, 2025

Related U.S. Application Data
(60) Provisional application No. 63/442,972, filed on Feb. 2, 2023.

(51) Int. Cl. A61B 8/10; A61B 8/00

(52) U.S. Cl. CPC A61B 8/10; A61B 8/4272; A61B 8/4483; A61B 8/485; A61B 8/5223

(57) ABSTRACT

The present disclosure provides systems and methods for characterizing and predicting an anatomical feature, and predicting the progression of a condition of the anatomical feature, based on a signal from an ultrasonic transducer. For example, the systems and methods disclosed herein can be utilized to characterize and predict a condition such as myopia based on an elasticity of an anterior sclera.

### CROSS-REFERENCE TO RELATED APPLICATIONS

This application claims the benefit of and priority to International Patent Application No. PCT/US2024/013825 filed Jan. 31, 2024, and U.S. Provisional Application No. 63/442,972 filed Feb. 2, 2023, the contents of each of which are incorporated by reference in their entirety for any and all purposes.

### STATEMENT OF FEDERALLY FUNDED RESEARCH

This invention was made with government support under grant number EB028084 awarded by National Institutes of Health. The government has certain rights in the invention.

### TECHNICAL FIELD

The present technology relates to systems and methods for characterizing a state of an anatomical structure based on ultrasonic signals. For example, an ultrasonic emitter-receiver pair can characterize a mechanical feature (e.g., elasticity, viscoelasticity, or microstructure) of an anterior sclera to predict a progression of myopia.

### BACKGROUND

The following description of the background of the present technology is provided simply as an aid in understanding the present technology and is not admitted to describe or constitute prior art to the present technology.

Aspects of a subject's visual acuity may be characterized by a refraction test, wherein an optometrist or other tester lenses an input to the eye according to lenses having varying refractive indices. The characterization of the refraction test can provide a nature of a condition, such as myopia (near-sightedness) or hyperopia (farsightedness). Further, the characterization can quantify the condition, such as according to a diopter scale (e.g., from −1.0 to −2.0). However, some eye conditions such as myopia may progress according to behavioral or environmental factors of a subject, along with structural factors, related to a structure of anatomical features of the eye. Thus, while the refraction test may indicate a current visual acuity of a subject, it may be less strongly correlated with future outcomes, such as a progression of the condition. For example, the visual acuity may be less predictive than the structural features alone, or the structural features in combination with the visual acuity. However, some aspects of the structural features may not be readily observed aside from, for example, a post-mortem examination or vivisection, which may limit their application. Furthermore, although various therapeutic agents or procedures may address a condition of the eye, a treatment response may correspond to structural condition, in addition to or instead of a visual acuity, such that two subjects having a same visual acuity can have varying suitability for a particular procedure.

Accordingly, there is an urgent need for prognostic methods that employ ultrasonic imaging to characterize an anatomical feature of an eye, and predict future outcomes based on the current state thereof. Ultrasonic imaging can generate data maps depicting a representation of a characteristic of an anatomical feature. The data maps can represent a non-real-space image including various characteristics of the anatomical feature, such as density, phase shift, time delay, elasticity, attenuation, or so forth. Application of ultrasonic imaging of the eye has been limited according to available systems, methods and devices.

### SUMMARY OF EMBODIMENTS OF THE DISCLOSURE

In one aspect, an ultrasound probe is provided. The ultrasound probe includes a transducer to generate ultrasonic pressure waves at an acoustic frequency to penetrate the surface of the eye to a depth of an anatomical structure of the eye. The ultrasound probe includes a biocompatible end effector to contact a surface of the eye to propagate the ultrasonic pressure waves from the transducer to the eye. The ultrasound probe includes a sensor to detect a return pattern of the ultrasonic pressure waves, the return pattern indicative of a mechanical feature of the anatomical structure.

In some embodiments, the biocompatible end effector is configured to directly contact the surface of the eye at a terminal end.

In certain embodiments, the biocompatible end effector includes a longitudinal dimension between the transducer and the terminal end equal to a distance to a focal spot corresponding to the transducer, less a distance from the anatomical structure to the surface of the eye. The biocompatible end effector can include a conformal material to conform to the surface of the eye. The biocompatible end effector can include an ultrasound transmission gel along the terminal end.

In any of the preceding embodiments of the ultrasound probe, the anatomical structure can be or include the anterior sclera.

In any of the preceding embodiments of the ultrasound probe, the acoustic frequency can be in a range that is, for example, 1 kHz or higher, such as between about 20 kHz and about 200 MHz. In certain example embodiments, the acoustic frequency may be, for example, between about 48 MHz and about 120 MHz or between about 40 MHz and about 130 MHz. In other examples, the acoustic frequency may be at least about 1 MHz or higher. In yet other examples, the acoustic frequency may be, for example, between about 20 MHz and about 180 MHz, or between about 40 MHz and about 160 MHz, or between about 40 MHz and about 140 MHz, or between about 60 MHz and about 120 MHz, or between about 80 MHz and about 100 MHz, or between about 40 MHz and about 140 MHz, or between about 30 MHz and about 150 MHz.

In one aspect, the present disclosure provides a system including a transducer, a biocompatible end effector, and one or more processors. The transducer can generate ultrasonic pressure waves at an acoustic frequency to penetrate the surface of the eye to a depth of an anatomical structure of the eye. The transducer can detect a return pattern of the ultrasonic pressure waves, the return pattern indicative of a mechanical feature of the anatomical structure. The biocompatible end effector can propagate the ultrasonic pressure waves between the transducer and the surface of the eye. The one or more processors can analyze the return pattern to characterize the mechanical feature of the anatomical structure.

In some embodiments, the anatomic structure is the anterior sclera. The mechanical feature can include an elasticity of the anterior sclera. The one or more processors can, based on the characterization, predict a progression of myopia.

In any of the preceding embodiments, the characterization can be based on a plurality of parameters determined or reviewed by the one or more processors. The parameters can include a sphere equivalent refractive error of the eye; an axial length of the eye; a maximum speed of a shear wave determined from the return pattern; a mean speed of the shear wave determined from the return pattern; a standard deviation of the shear wave speed determined from the return pattern; an effective scatterer diameter determined from the return pattern; an effective acoustic concentration determined from the return pattern; an anterior chamber depth of the eye; u parameter of a Homodyned-K (HK) distribution determined from the return pattern; a parameter of the HK distribution.

In any of the preceding embodiments, the one or more processors can receive a plurality of longitudinal return patterns, the longitudinal return patterns corresponding to a longitudinal time of greater than one year. The one or more processors can predict, based on the plurality of longitudinal return patterns and the longitudinal time, a future progression of the condition of the eye.

In any of the preceding embodiments, the one or more processors can determine, based on the return pattern, that the transducer should be reoriented. The one or more processors can provide, via a user interface, an indication that the transducer should be reoriented.

In any of the preceding embodiments, the one or more processors can compare the mechanical feature to a mechanical feature threshold and provide, via the user interface, an indication of the mechanical feature, based on the comparison.

In any of the preceding embodiments, the mechanical feature can include a plurality of elasticities corresponding to a plurality of loci of the anatomical structure.

In any of the preceding embodiments, the one or more processors can predict a progression of a condition of the eye and provide, via the user interface, the prediction.

In any of the preceding embodiments, the one or more processors can, during a first mode of operation, cause the transducer to receive a first portion of the return signal from ambient sources. During a second mode of operation temporally offset from the first mode of operation, the one or more processors can cause the transducer to generate the ultrasonic pressure waves and receive a second portion of the return signal.

In certain embodiments, the one or more processors generate a first portion of a data map from the first portion of the return signal, the first portion of the data map corresponding to a stationary position of the transducer relative to the eye. The one or more processors can further generate a second portion of a data map from the second portion of the return signal, the second portion of the data map corresponding to a moving position of the transducer relative to the eye.

In one aspect, the present disclosure provides a method. The method can include characterizing a mechanical feature of an anatomical structure of the eye of a patient, using the ultrasonic probe or the systems disclosed above.

In some embodiments, the characterization of the anatomical structure is predictive of a progression of myopia.

In any embodiments of the methods disclosed herein, the patient can be human.

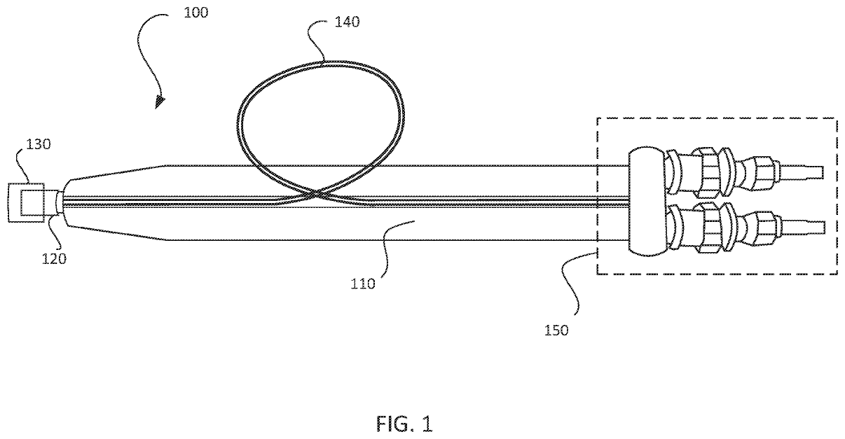

FIG. 1 is a depiction of an ultrasound probe, according to an illustrative example of the present disclosure.

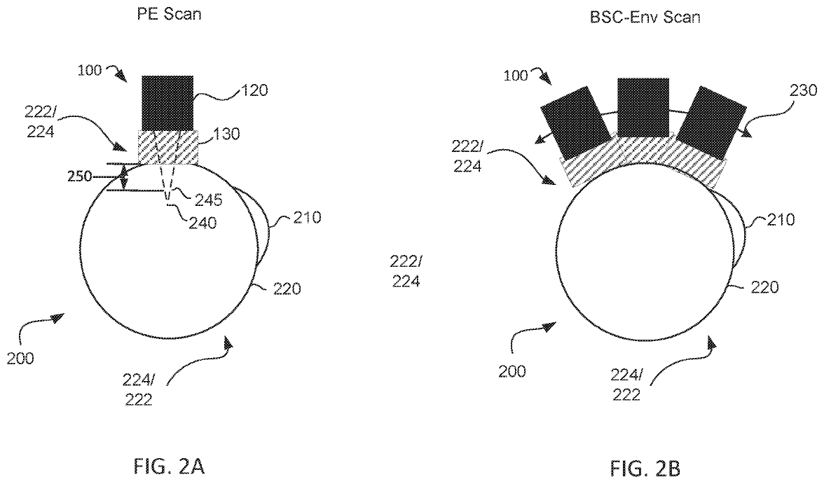

FIGS. 2A-2B are depictions of an ultrasound probe in relation to an eye, according to an illustrative example of the present disclosure.

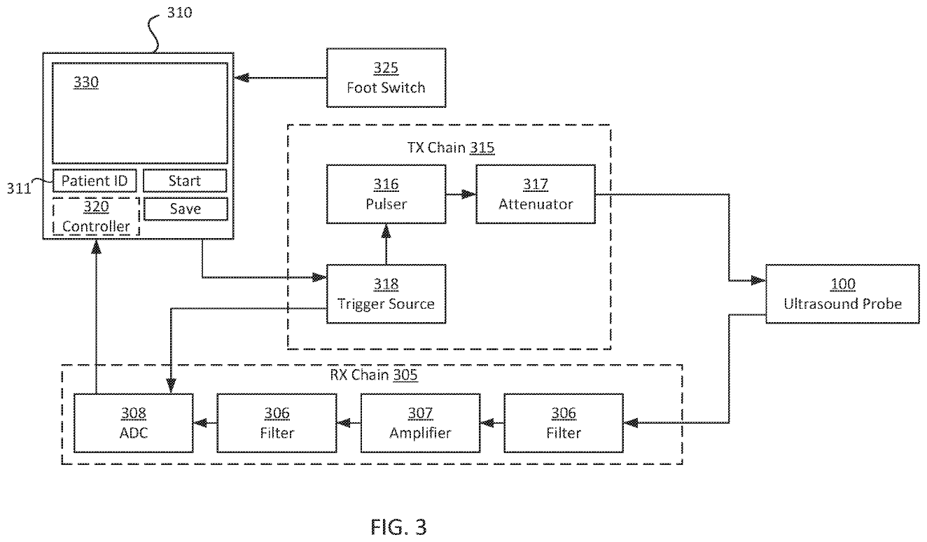

FIG. 3 is a block diagram of a system to characterize or predict a progression of a condition of an eye, according to an illustrative example of the present disclosure.

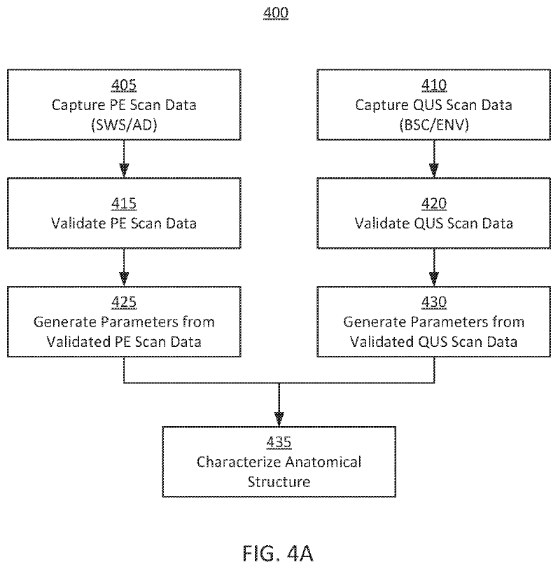

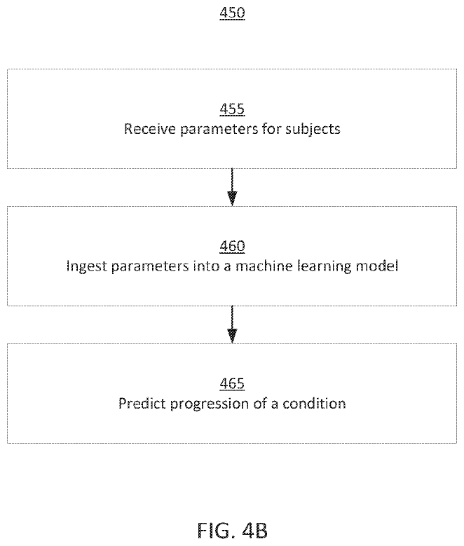

FIG. 4A is a flowchart for a method of characterizing an anatomical structure, according to an illustrative example of the present disclosure.

FIG. 4B is a flowchart for a method of predicting a progression of a condition of an eye, according to an illustrative example of the present disclosure.

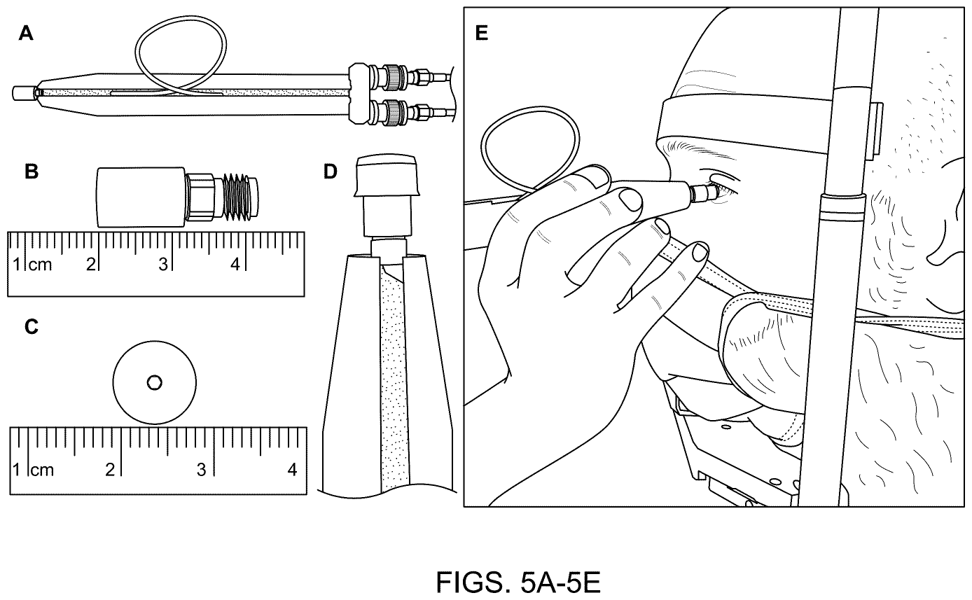

FIGS. 5A-5E depict the POC instrument for scanning the anterior sclera, according to an illustrative example of the present disclosure. FIG. 5A depicts the handheld “stylus” portion of the instrument. FIGS. 5B-5C depict the 80 MHz transducer. FIG. 5D depicts the custom-made silicone caps to protect the eye and provide the appropriate offset between the transducer and eye for obtaining high-quality RF data from the sclera. FIG. 5E depicts scanning the anterior-temporal sclera of a volunteer.

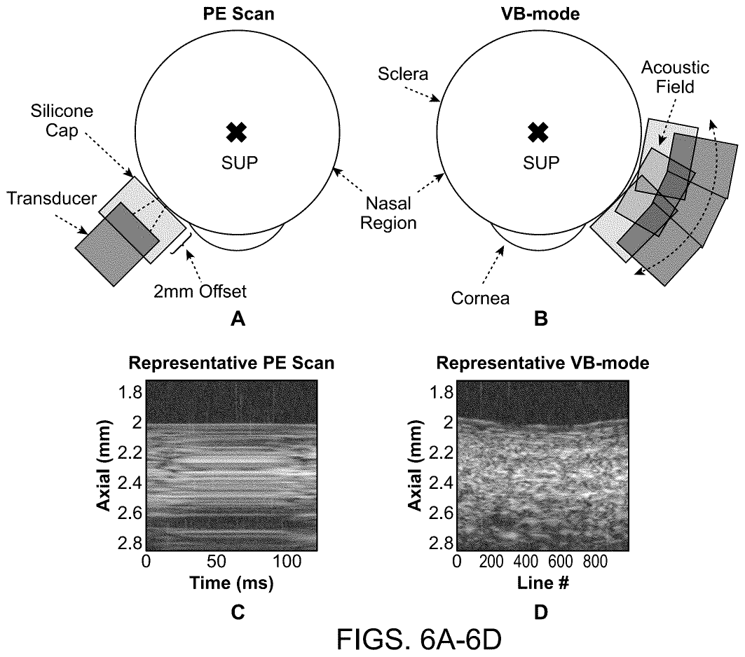

FIGS. 6A-6D depict protocols for scanning the anterior sclera with the POC instrument, according to an illustrative example of the present disclosure. FIG. 6A depicts PE scans performed by holding the transducer in place and collecting RF data from the same location of the sclera. FIG. 6B depicts VB-modes collected by translating the transducer in across the sclera. Representative frames from a successful PE scan and VB-mode scan are provided at FIGS. 6C and 6D, respectively.

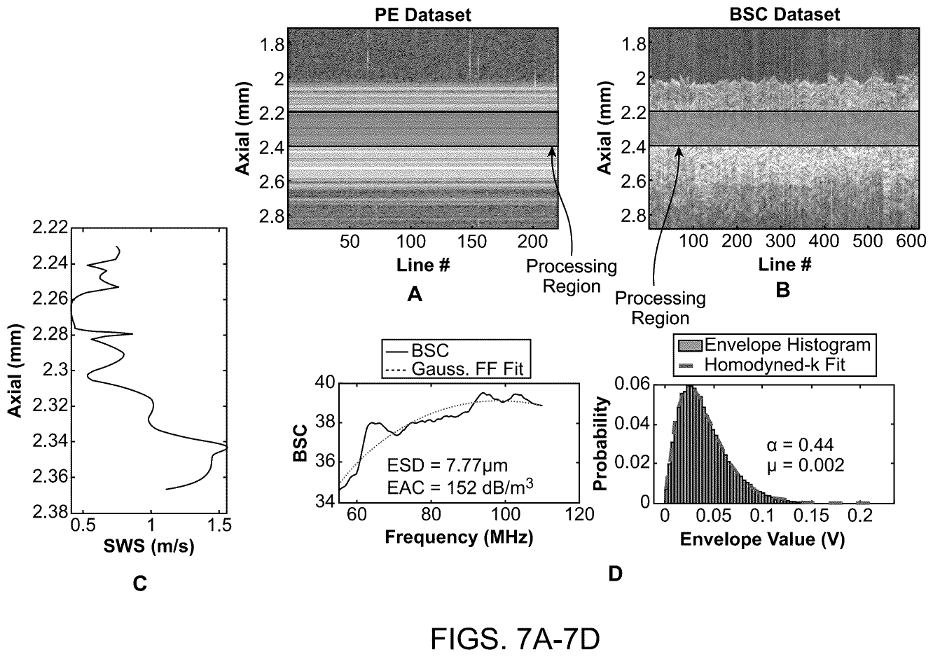

FIGS. 7A-7D depict examples of PE and BSC datasets generated with the semi-automated algorithm, according to an illustrative example of the present disclosure. FIG. 7A depicts a PE dataset for SWS estimation. FIG. 7B depicts a BSC dataset created by combining uncorrelated RF lines from multiple echo frames. FIG. 7C depicts depth-dependent SWS computed from the PE dataset in FIG. 7A. FIG. 7D depicts BSC with scattering model fit (top) and histogram of envelope values with HK probability density function fit (bottom) computed from the BSC dataset of FIG. 7B.

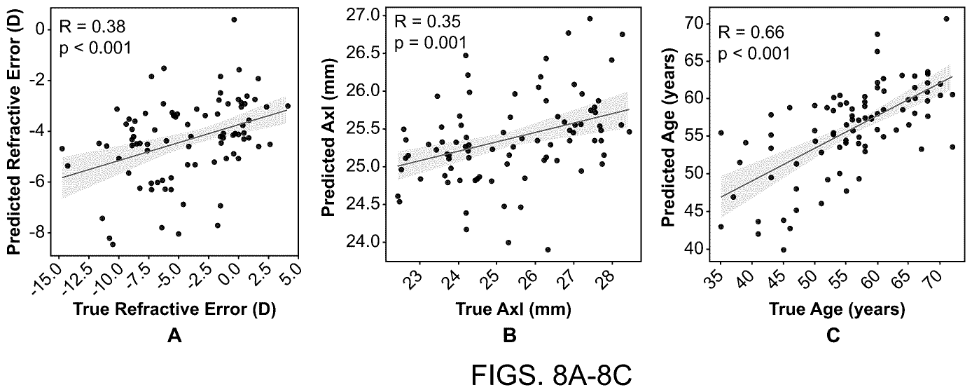

FIGS. 8A-8C depict multilinear regression of QUS parameters with refractive error (at FIG. 8A), axial length (at FIG. 8B), and patient age (at FIG. 8C), according to an illustrative example of the present disclosure. The shaded region corresponds to the 95% confidence interval.

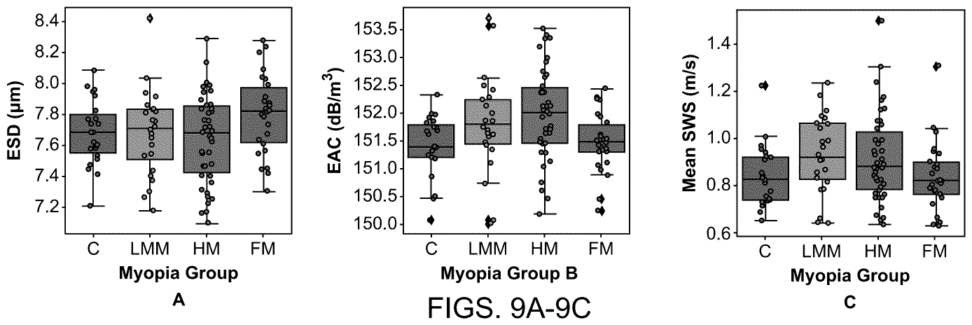

FIGS. 9A-9C depict box plots of QUS parameters, according to an illustrative example of the present disclosure. Particularly, ESD at FIG. 9A, EAC at FIG. 9B, and mean SWS at FIG. 9C.

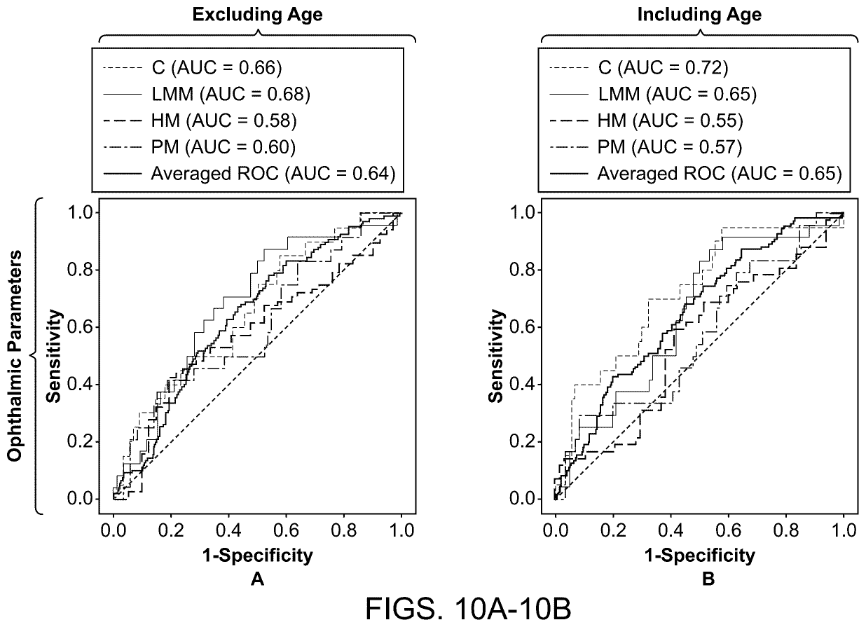

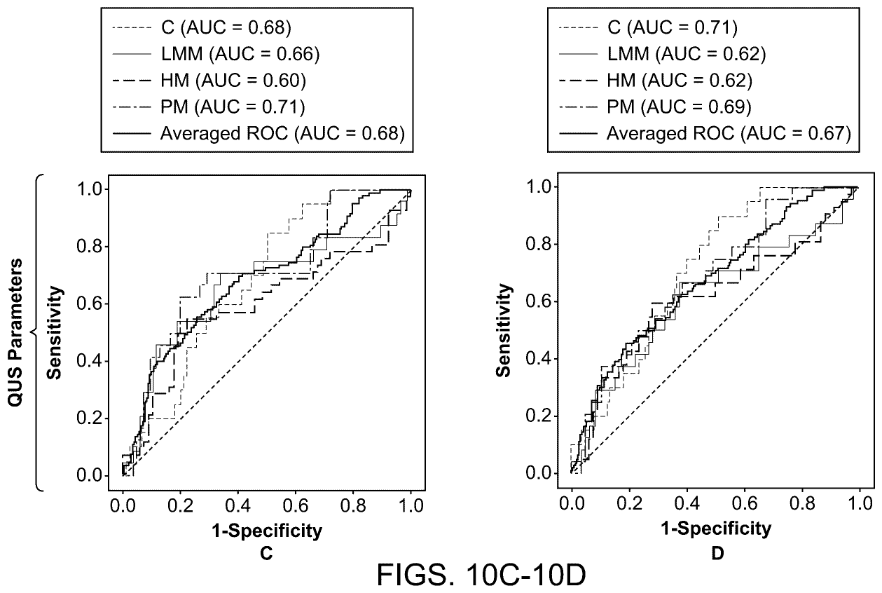

FIGS. 10A-10D depict ROC curves for a machine learning classifier evaluated using a one-vs-rest paradigm with (top row) ACD, CCT, IOP, and K as input features and (bottom row) QUS parameters as input features, according to an illustrative example of the present disclosure. Patient age was (left column) excluded or (right column) included with input features.

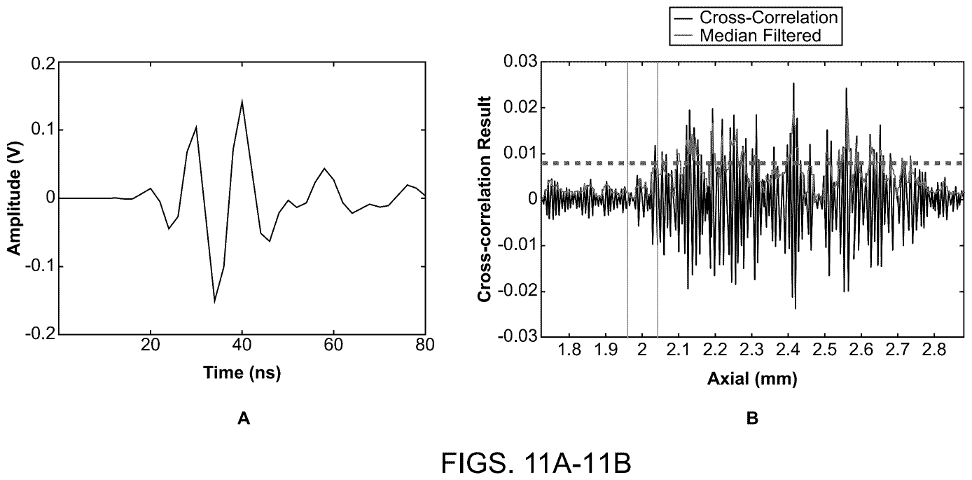

FIGS. 11A-11B depict reference pulse of the 80 MHz transducer at FIG. 11A, and cross-correlation of an RF line with the reference pulse (black curve) and the result of median filtering (red curve) at FIG. 11B, according to an illustrative example of the present disclosure. The dashed horizontal green line indicates the threshold for identifying the sclera surface. RF lines depicted where the detected sclera surface resided within the axial range specified by the vertical blue lines were retained for further processing.

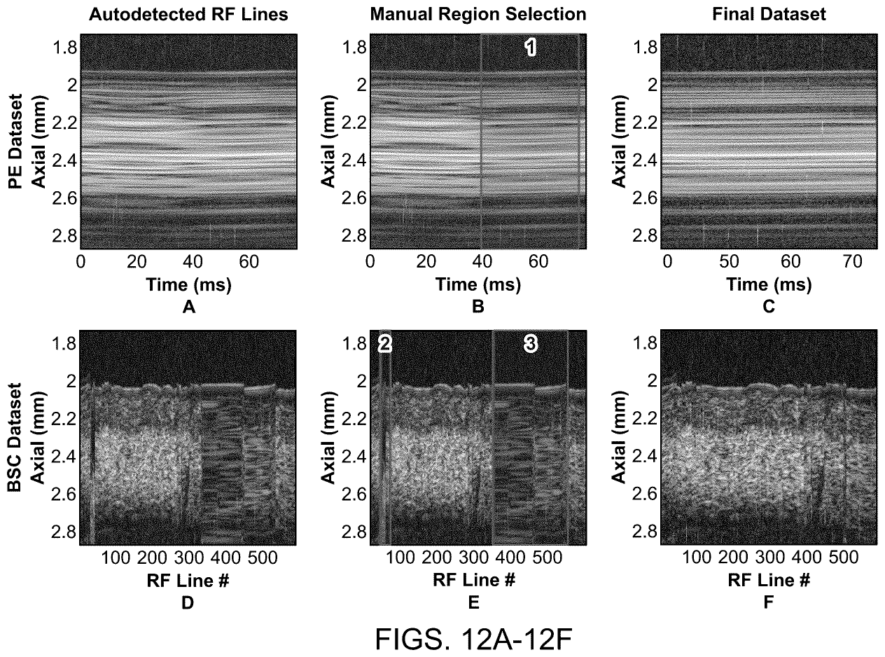

FIGS. 12A-12F provide examples of manual region exclusion for PE (top row) and BSC (bottom row) datasets, according to an illustrative example of the present disclosure. The first column contains representative datasets generated by the automated algorithm at FIGS. 12A, 12D. The middle column shows the regions manually identified for retention for PE processing at FIG. 12B or exclusion from BSC processing at FIG. 12B. The last column are the final PE at FIG. 12C and BSC datasets at FIG. 12F output for further QUS processing.

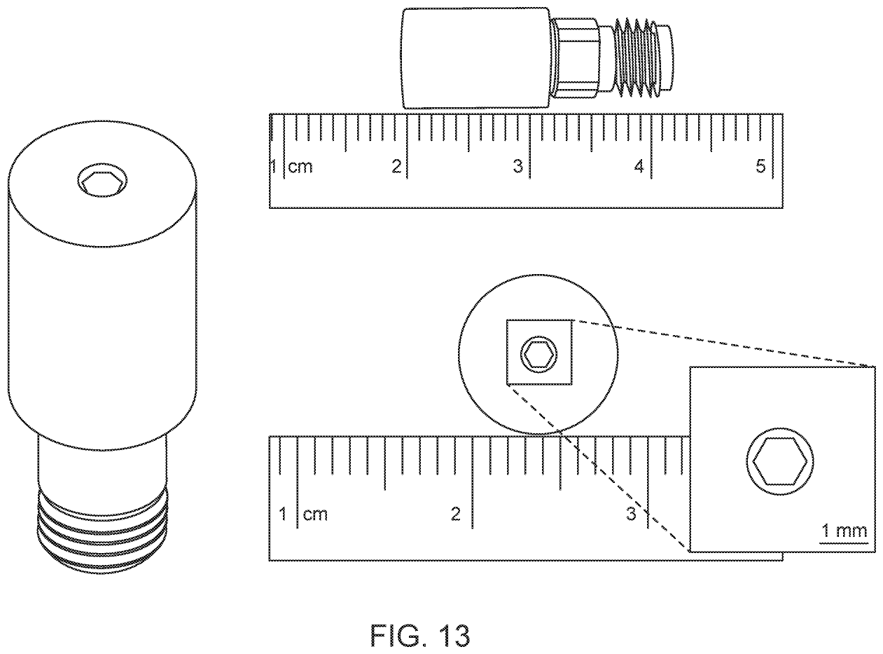

FIG. 13 depicts the 80 MHz transducer, according to an illustrative example of the present disclosure. The inset depiction is a zoomed in view of the 1.2 mm diameter active element of the transducer.

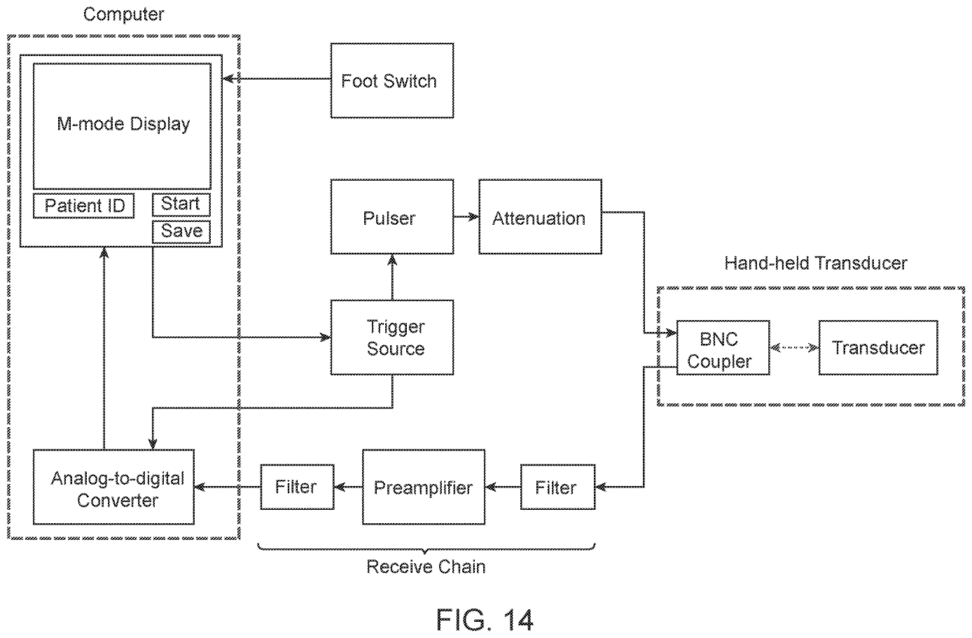

FIG. 14 is a block diagram of the point-of-care device, according to an illustrative example of the present disclosure. Components enclosed within the red dashed box are part of a desktop computer. The blue dotted box represents components within the handheld transducer. In example embodiments, the length of the coaxial cable represented by the red dotted arrow has a significant effect on the pulse generated by the transducer.

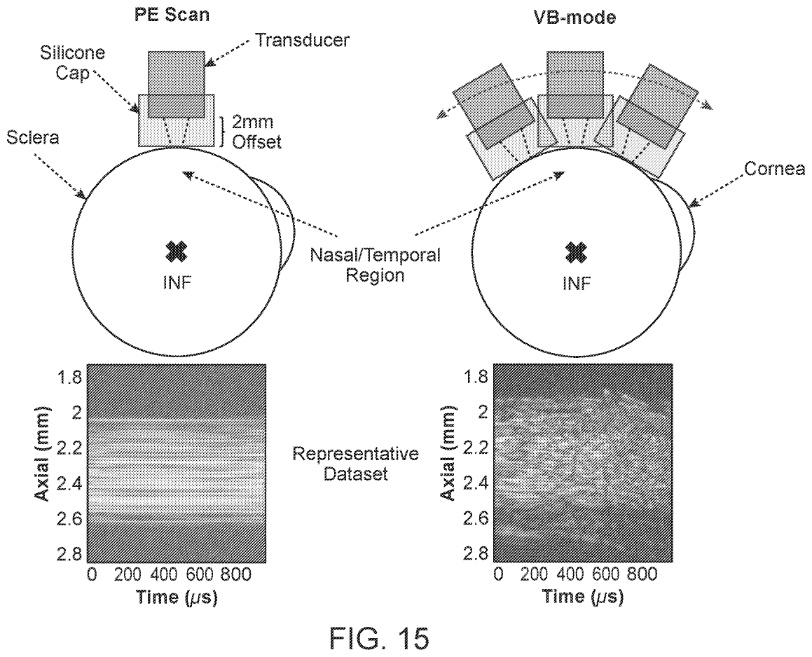

FIG. 15 illustrates the passive elastography (PE) scans and virtual B-modes, according to an illustrative example of the present disclosure. The bottom images are representations of the data collected for each scan type. (left) During a PE scan, the transducer is held in place to acquire motion-mode data at one location. (right) The transducer is swept back and forth over ≈1 mm to collect independent radio frequency echo lines and create a virtual B-mode.

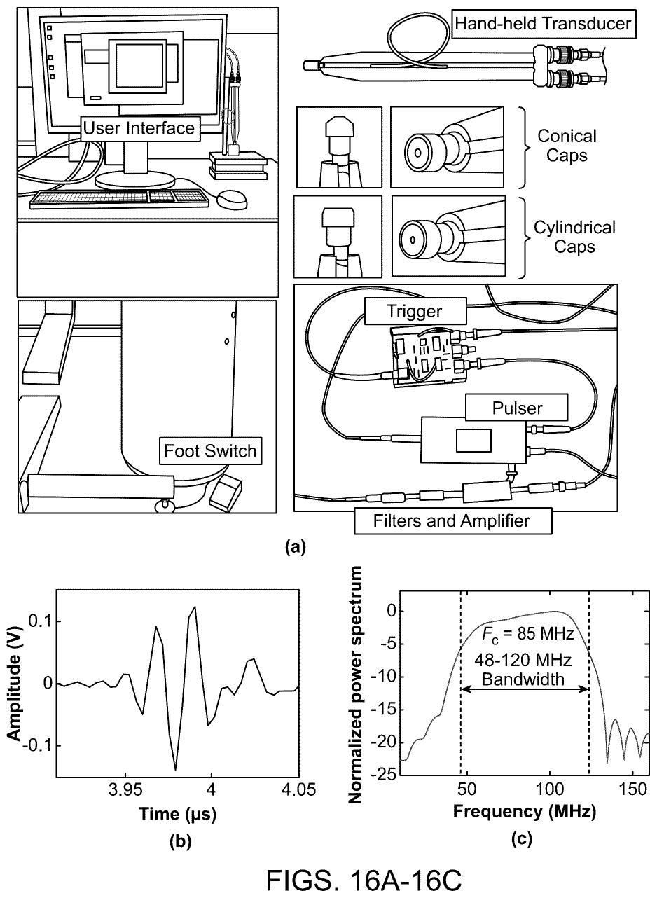

FIG. 16A depicts a point-of-care system, according to an illustrative example of the present disclosure. FIG. 16B depicts representative ultrasound signal generated by the transducer. FIG. 16C depicts representative normalized power spectrum of the pulse in FIG. 16B. The center frequency of the transducer is the center of the −6 dB pulse bandwidth.

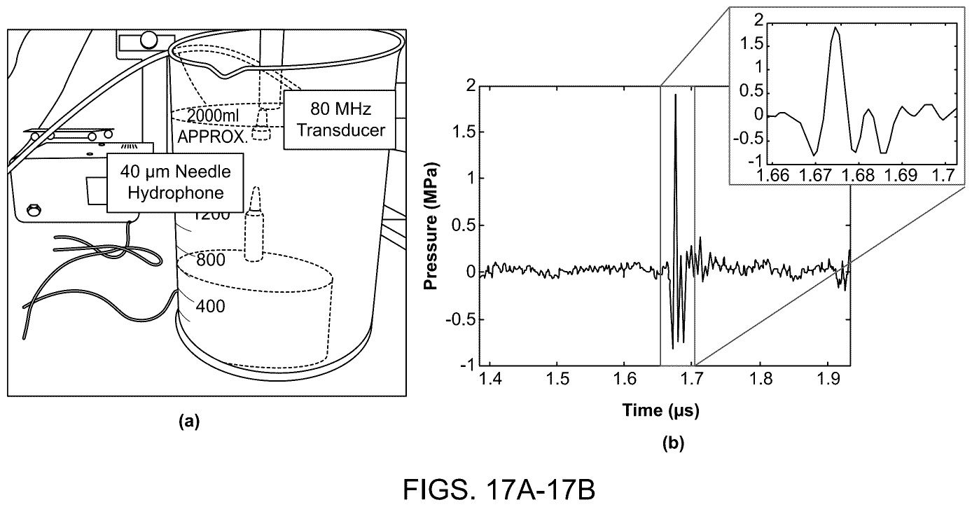

FIG. 17A depicts an exposimetry set-up to measure pressure field generated by point-of-care device, according to an illustrative example of the present disclosure. FIG. 17B is a recording of the maximum measured pressure signal, according to an illustrative example of the present disclosure.

FIG. 18 (left) is an in vivo measurement of the temporal anterior sclera of a healthy volunteer, according to an illustrative example of the present disclosure. FIG. 18 (right) is a representative motion-mode and virtual B-mode images of data deemed suitable for PE or QUS processing, according to an illustrative example of the present disclosure. Acronyms include M-mode, motion-mode; PE, passive elastography; QUS, quantitative ultrasound.

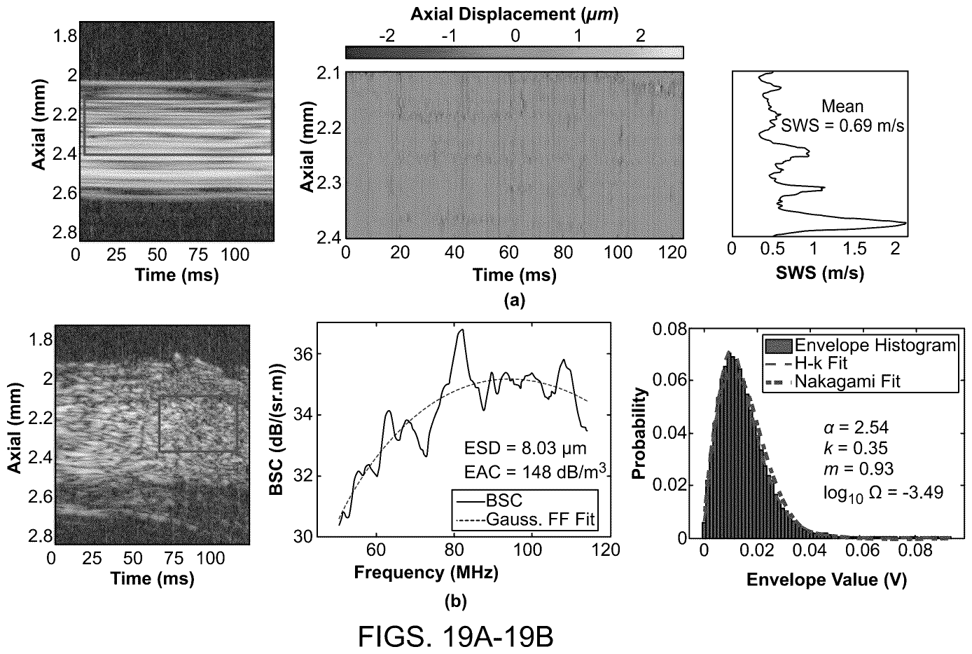

FIG. 19A depicts results of passive elastography processing of in vivo data, according to an illustrative example of the present disclosure. The map of axial displacements corresponds to the region bounded by the red box in the motion-mode image. Passive elastography processing provides depth-dependent estimates of shear-wave speed. FIG. 19B depicts results of quantitative ultrasound processing of in vivo data. Backscatter coefficient parameters and envelope statistics were computed from the region enclosed by the red box in the virtual B-mode image. Acronyms include BSC, backscatter coefficient; EAC, effective acoustic concentration; ESD, effective scatterer diameter; SWS, shear wave speed.

### DETAILED DESCRIPTION

It is to be appreciated that certain aspects, modes, embodiments, variations and features of the present methods are described below in various levels of detail in order to provide a substantial understanding of the present technology. The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, tissues, anatomical features, methods, reagents, compounds, compositions or biological systems, which can, of course, vary. It is also to be understood that the terminology used herein is for the purpose of describing particular embodiments only, and is not intended to be limiting.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the terms “approximately,” “about,” “substantially,” and similar terms in reference to a number or value is generally taken to include numbers or values that fall within a range of 1%, 5%, or 10% in either direction (greater than or less than) of the number or value unless otherwise stated or otherwise evident from the context (except where such number would be less than 0% or exceed 100% of a possible value).

As used herein, the terms “individual”, “patient”, or “subject” are used interchangeably and refer to an individual organism, a vertebrate, a mammal, or a human. In a preferred embodiment, the individual, patient or subject is a human.

For purposes of reading the description of the various embodiments, the following descriptions of the following descriptions of the sections of the specification and their respective contents may be helpful:

Section A describes systems and methods for the characterization of the anatomical structures of the eye. The characterization can be employed to predict a future state of the anatomical structures, such as a future structural condition or a change in visual acuity corresponding to such a structural condition.

Section B describes a clinical example of an application of the systems and methods of Section A to a sample population.

Section C describes a clinical example of an application of the systems and methods of Section A to another sample population.

Section D describes selected example systems, methods, and devices of the present disclosure.

### A. Characterization of the Anatomical Structures of the Eye.

Referring now to FIG. 1, an ultrasonic probe 100 is provided, according to an illustrative example of the present disclosure. The ultrasonic probe 100 can exchange signals with a surface of an eye. For example, the ultrasonic probe 100 can generate signals for transmission to the surface of the eye, and receive a return pattern therefrom, the return pattern indicative of a character (e.g., state, condition, or progression thereof) of the eye. The signal generated or conveyed by the ultrasonic probe 100 can include an ultrasonic pressure wave (e.g., a signal having components between 1 kHz and 200 MHz). The signal can include an acoustic frequency, amplitude, or directionality to cause the signal to penetrate the surface of the eye to a depth of an anatomical structure, interact with the anatomical structure, and reflect a return pattern indicative of characteristics of the anatomical structures of the eye (e.g., of the sclera). For example, the return pattern can be indicative of a mechanical feature such as an elasticity or other mechanical property or microstructure of one or more loci of the eye. In some embodiments or instances, the return pattern can be or include a return pattern from a source other than the ultrasonic probe 100. For example, the source can include ambient sources such as respiratory or cardiac system motion (e.g., blood vessels in the eye), or other environmental stimuli.

The ultrasonic probe 100 includes a body 110 to couple the various components of the ultrasonic probe 100 (e.g., mechanically or electrically). The body 110 can be grasped and manipulated by a user to align a transducer 120 with an eye. The body 110 can receive, at one terminal end, the transducer 120, and receive, at another terminal end, the connector 150. Either of the transducer 120 or the connector 150 can be selectively or fixedly coupled with the body. For example, the transducer 120 can be received into the body via threads. The transducer 120 can be selectively removable (e.g., for maintenance or to select a different transducer 120). For example, a transducer 120 having a different frequency, amplitude, directionality, or other characteristic can be connected for some measurements. Likewise, the connector 150 can be fixedly or selectively coupled with other elements of an associated system, such as the RF medium 140 coupled with a computing device. For example, the connector 150 can include a Bayonet-Neill-Concelman (BNC) connector, threads, welds or other connections for one or more portions thereof.

In some embodiments, the connector 150 can include an impedance-controlled connection (e.g., 50Ω, 90Ω, or 100Ω). The impedance-controlled connection can match a characteristic impedance of either or both of an RF medium 140 between the transducer 120 and the connection 150, or another RF medium between the connection and other components of the associated system. The body 110 can include an RF transparent material to avoid attenuating signals exchanged along the RF medium 140, or can include RF shielding to shield from unintended signal paths. The mechanical length of the body 110 can match or differ from an electrical length of an RF medium 140. For example, a mechanical length can be selected to aid an ergonomic grasp of a user, and the electrical length can be selected to aid signal transmission. The electrical length can correspond to a frequency of interest (e.g., of the transducer 120). For example, the body 110 can include openings to extend (e.g., coil, serpentine, etc.) the RF medium 140 to extend a full, half, or quarter-wave distance of a center frequency of the transducer 120. Such a distance can correspond to the distance of the RF medium 140 between the transducer 120 and the connector 150 or further include another portion of the RF medium (e.g., a portion extending between the connector 150 and further system elements disclosed herein). For example, a first electrical distance can correspond to a distance between the transducer 120 and a discontinuity at the connector 150, and a second electrical distance can correspond to a distance between the connector 150 and a transmit (TX) or receive (RX) chain component.

In some embodiments, the body 110 can receive or otherwise couple with further components. For example, in some embodiments, the body 110 can include or otherwise couple with an engageable switch configured to engage the transducer 120. The engageable switch can be provided instead of or in addition to (e.g., in series with) another switch, such as a foot pedal control. In some embodiments, the body 110 can include or otherwise couple with a portion of a user interface configured to indicate an orientation of the ultrasonic probe 100 (e.g., that the probe should be reoriented). For example, the indication can include an audible, visual, or haptic component (e.g., from a speaker, light emitting diode (LED), or haptic motor of the user interface). The portion of the user interface can be communicatively coupled with a computing device configured to present an indication based on a receipt of scan data from the ultrasonic probe 100.

The ultrasonic probe 100 includes an ultrasonic transducer 120 to generate or receive ultrasonic signals. An ultrasonic signal can refer to or include a pressure wave exceeding human hearing (e.g., 20 kHz). For example, ultrasonics can include signals in a kHz range, low megahertz range (e.g., from 1 MHz to 20 MHz, which may also be referred to as a “medical ultrasound” range, for their use in medical imaging), or frequencies exceeding twenty MHz (e.g., between 48 and 120 MHz, such as signal having a center frequency between 70 and 105 MHz). The transducer 120 can be configured to emit an ultrasonic signal configured to generate a pressure wave in an end effector 130 of the ultrasonic probe 100 to transfer the pressure wave to the surface of the eye.

The transducer 120 can be configured to receive a return pattern. The return pattern can refer to or include a reflection of a signal generated from the transducer 120, or a return from signals derived from any of various further sources (e.g., cardiac, respiratory, or environmental activity, collectively, ambient sources). For example, during a passive elastography (PE) scan, the transducer 120 can receive a signal indicative of a shear wave or axial displacement based on a signal returned to the transducer 120 from ambient sources. During a quantitative ultrasound (QUS), the transducer 120 can receive a reflection of a transmitted signal, and determine parameters based on a transformation between an emitted and returned signals. Such parameters can include a backscatter coefficient (BSC), effective acoustic concentration (EAC), effective scatter diameter (ESD), or various values of a Homodyned-K distribution, such as an envelope, a parameter, u parameters or so forth.

In some embodiments, the transducer 120 can include an emitter-transducer 120 to generate the pressure wave, and a sensor-transducer 120, separate from the emitter-transducer 120, to sense a return pattern from the eye. In some embodiments, the transducer 120 can include a same element to generate the pressure wave and receive the return pattern (e.g., the emitter-transducer 120 and sensor-transducer 120 can be a same component of the transducer). In some instances, the transducer 120 may not engage an emitter-transducer 120 during a scan. The sensor-transducer 120 can be referred to as a “sensor” in any case.

The transducer 120 can generate a signal having a focal spot (e.g., an area or volume surrounding or otherwise proximal to a focal point. The focal spot can include data having a focus greater than a threshold value. For example, a focal spot can be disposed somewhat shallower than a focal point, wherein the spot is substantially in focus, and includes a greater area than at a depth of the focal point. The focal spot can be a fixed distance in front the of transducer 120. For example, the transducer can have a focal spot about 2 mm forward of the emitter-transducer 120 (e.g., wherein the focal point is somewhat deeper, such as, for example, about 2.4 mm).

An end effector 130 coupled with the transducer 120 can space the transducer 120 the focal spot distance from various anatomical structures. For example, the end effector 130 can extend a distance less than the focal distance from the transducer 120, such that upon a direct contact between the end effector 130 and a surface of an eye, the focal spot extends beyond the surface of the eye to a depth of the anatomical structure. For example, the anatomical structure can include the anterior sclera (e.g., the depth can be equal to or less than 1 millimeters (mm), such as 500 micro-meters (μm), 200 μm, or 100 μm).

The end effector 130 can be a biocompatible end effector 130 (e.g., configured for direct contact with the surface of the eye). For example, the end effector 130 can include a conformal material having a durometer hardness of between 00 and 50 (e.g., Eco-Flex™ 00-50). The end effector 130 may form, in a relaxed state, a generally cylindrical or conical shape configured to deform upon contact with the surface of the eye. In some embodiments, the end effector 130 can include an ultrasonic gel or other coupling fluid to acoustically couple the end-effector 130 with the surface of the eye. Such a coupling fluid can lower or otherwise control attenuation at an interface between the end-effector 130 and the surface of the eye. In some embodiments, the ultrasonic gel of the end effector 130 can include (or can otherwise be employed in combination with) a topical anesthetic for the eye, such as tetracaine or proparacaine.

The end effector composition can be selected according to a frequency of operation of the transducer 120. For example, some materials may be relatively opaque to MHz range signals (e.g., signals having a center frequency between 70 and 105 MHz, and bandwidth extending between 48-120 MHz). The selected material of the end effector can vary according to a selected transducer 120. For example, a transducer 120 configured for operation at or below 30 MHz can include a different material to manage attenuation. In some embodiments, the combination of a transducer 120 and an end effector 130 can be referred to as a head assembly. Various head assemblies can correspond to different scan depths and focal spots or points, such that a material of the end effector 130 can be selected according to an attenuation of the transducer 120, and a geometry of the end effector 130 is selected according to a focal point or sport (e.g., a depth thereof, wherein the depth of the focal point can accord with the frequency of the transducer 120, such that high frequency transducers 120 may include lower penetration depths than lower frequency transducers 120 at a given power level).

The ultrasonic probe 100 includes an RF medium 140 to couple a connector 150 to communicatively couple with other system portions. For example, the RF medium 140 can include a wired or wireless connection (e.g., an impedance controlled coaxial connection). In some embodiments, the RF medium can include a same or different RF medium 140 for a transmit (TX) chain and a receive (RX) chain (e.g., may be configured for temporally orthogonal TX/RX operation). For example, as depicted, a separate coaxial connection can correspond to the respective RX and TX chain. In some embodiments, the RF medium 140 can include a single-ended medium. In some embodiments, the RF medium 140 can include a differential medium. Accordingly, the connector 150 can include a same or separate RX/TX medium, differential connection 150, and so forth. The connector 150 can couple the transducer to a signal generator (e.g., the pulser 316 of FIG. 3) or receiver (e.g., the RX chain 305 of FIG. 3). Such coupling, like other references to couplings or connections herein, may include direct coupling (connecting without intervening elements) or indirect coupling (e.g., coupling with one or more intervening elements).

Referring now to FIGS. 2A-2B, depictions of an ultrasonic probe 100 in relation to an eye 200 are provided, according to an illustrative example of the present disclosure. The ultrasonic probe 100 (also referred to as an ultrasound probe, without limiting effect) is illustrated disposed along an anterior sclera 220 of the peripheral regions of the eye (e.g., the nasal region 222, proximal to a nose of a subject relative to the cornea 210, or the temporal region 224, proximal to a temple of a subject relative to the cornea 210). The position of the ultrasonic probe 100 can correspond to a measurement type. The transducer 120 can resolve to a focal point 240 to cause a focal spot 245 a desired depth 250 into the eye (e.g., corresponding to an anatomical feature thereof). In various embodiments, the depth 250 can extend to various portions of the anterior sclera 220. The depth 250, like other features of the figures, is not intended to be drawn to scale. Indeed, various aspects of the figures provided herein may be increased or decreased for clarity or reproducibility. In some embodiments, a same transducer 120 can be employed for various operations such as the PE scan of FIG. 2A and the QUS scan of FIG. 2B. The focal point 240 and spot 245 are omitted from FIG. 2B for clarity. However, such as focal point, for a QUS scan can refer to either of both of a focus for a signal conveyed to the transducer 120 from the eye 200, or a signal returned from the eye 200 to the transducer 120.

Referring particularly to FIG. 2A, an ultrasonic probe 100 is provided relative to an eye 200 incident to a PE (passive elastography) scan. During such a scan, the ultrasonic probe 100 (e.g., the transducer 120 thereof) may be maintained in a stationary position along the sclera 220 of the eye 200. According to the stationary position, a return signal can provide in indication of the state of various anatomical features of the eye 200 (e.g., tissues of the anterior sclera 220). For example, ambient sources can generate signals to interact with the anatomical structure of the eye 200, and return such signals to a sensor (e.g., the transducer 120, via the end effector 130). The ultrasonic probe 100 can pass such returned signals to other portions of the systems disclosed herein (e.g., the system 300 of FIG. 3) for processing to determine various characteristics of the anatomical features of the eye 200.

In some embodiments, the ultrasonic probe 100 is coupled with a system to automatically trigger the collect data based on a receipt of data indicative of an orientation of the ultrasonic probe 100 with respect to the sclera 220. For example, a pulse repetition frequency (PRF) or number of scan lines can be compared to a threshold level, and the trigger can chapiter data based on data exceeding the threshold levels. For example, a PRF threshold can be in a kHz range, such as between 4-8 kHz (e.g., about 6.6 kHz). A scan line quantity threshold can be greater than 100, such as 150.

In some embodiments, the ultrasonic probe 100 or other system portion includes or is coupled with an engageable switch to manually or semi-manually (e.g., in combination with the automatic trigger), collect data. Such a switch can be disposed on the ultrasonic probe 100 itself (e.g., a mechanical or capacitive button) or otherwise coupled with the ultrasonic probe 100 (e.g., a foot pedal switch). In some embodiments, a user interface integral to or otherwise coupled with the ultrasonic probe 100 can indicate when an automatic trigger is active, or when data is indicative of an engagement of a trigger by an operator of the ultrasonic probe 100.

The PE scan can be repeated along various portions of the eye. For example, the PE scan can be performed at one or more locations along the nasal region 222, the temporal region 224, the superior region, or the inferior region. In some embodiments, varying end effectors 130 may be employed for various regions. In some embodiments, one or more regions can be omitted according to a selected end effector 130. For example, some cylindrical end effectors 130 may exceed a clearance available for measuring the superior or inferior regions where the eyelid or other tissues interfere with such measurements.

Some quantitative ultrasound (QUS) techniques can generate additional parameters. Such parameters can include envelope statistics or backscatter scan parameters, which may be referred to generally as further QUS parameters, to distinguish from PE parameters determined according to a PE scan. During such a QUS scan, the ultrasonic probe 100 (e.g., the transducer 120 thereof) may be advanced along the sclera 220 during data measurement. Referring particularly to FIG. 2B, the advancement may proceed along a surface that is substantially perpendicular to the surface of the eye 200, such as the depicted arclength, or along a straight line which may approximate the arclength 230 for some distances of motion (e.g., 1 mm). For example, the end effector 130 can conform to a spacing between the transducer 120 and the eye 200 so as to maintain the focal spot 245 within a region of interest (e.g., depth 250) of the eye, while maintaining sonic coupling between the transducer 120 and the eye 200. The QUS can be performed at various locations of the eye 200, as discussed above with regard to the PE scan, such as the nasal region 222, the temporal region 224, the superior region, or the inferior region. Indeed, the various techniques or components (e.g., automatic or manual triggering) discussed with respect to FIG. 2A may be employed for the QUS depicted in FIG. 2B.

For the QUS of FIG. 2, the transducer can impart a signal to the eye 200, via the end effector 130. For example, the transducer can impart a signal having a focal spot 245 within the sclera 220, such that a return pattern indicates a transform of the imparted signal. The trigger can trigger automatically or manually, and can likewise provide an indication to a user indicating a validation of collected data. For example a number of distinct (e.g., independent or uncorrelated) RF echo lines can be received, and compared to a threshold attenuation relative to a transmitted signal. The number of lines can be compared to a threshold, such as a number greater than 50 lines (e.g., 75 lines). BSC and envelope statistics can be computed on valid, triggered data.

Referring now to FIG. 3, a block diagram of a system 300 to characterize or predict a progression of a condition of an eye 200 is provided, according to an illustrative example of the present disclosure. The ultrasonic probe 100 is coupled with an RX chain 305 to convey return signal data to a computing device 310 and a TX chain 315 to convey signals to the ultrasonic probe 100, the signals generated according to the computing device 310 (e.g., by engaging a pulser 316). The computing device 310 can include a controller 320 and a user interface such as a display, network connection, or so forth.

The RX chain 305 can include any number of filters 306 and amplifiers 307, (e.g., preamplifiers 307) to process a return signal. For example, the filters 306 and amplifiers 307 can amplify a signal of interest (e.g., a signal corresponding to a focal spot on an anatomical structure). The signal of interest can be identified according to a frequency. For example, the frequency can correspond a known source, such as blood flow through ocular vasculature or a signal generated by the transducer 120. The filters 306 and amplifiers 307 can condition the signal according to an input range of an ADC 308. For example, the ADC 308 can resolve information encoded according to an analog signal into digital information accessible to the computing device.

The TX chain 315 can include any number of signal generators (e.g., waveform generators, or conditioners such as phase and amplitude shifters). For example, the TX chain 315 can include a pulser 316 to generate a signal having a known amplitude and frequency, configured for transmittal along the RF medium 140 to the transducer 120, and onward to the end effector 130. An attenuator 317 or another signal conditioner can condition the signal. The conditioning can control an output level such as a temporal average intensity or a mechanical index. For example, the attenuator 317 can be configured to output a signal having a temporal average intensity of not greater than 50 mW/cm² or a mechanical index not greater than 0.23. In some embodiments, the attenuator can be substituted for an amplifier. For example, in embodiments including a pulser 316 receiving a signal having a temporal average intensity less than 50 mW/cm² or a mechanical index less than 0.23.

The TX chain 315 can include a trigger source 318 to trigger the output. For example, the trigger source 318 can be based on a determination of the computing device 310, or according to a selectively engaged switch, such as the depicted foot switch 325 or a switch of the ultrasonic probe 100. In some embodiments, the trigger source 318 can couple with both the TX chain 315 and the RX chain 305. For example, the trigger source 318 can trigger an output of a signal for conveyance to the eye 200, and to trigger the receipt of a return signal corresponding to a reflection thereof. In some embodiments, the trigger source 318 can provide the trigger to the RX chain 305 and TX chain 315 offset according to an offset time, to cause the RX chain 305 to receive a reflected instance of the signal applied to the TX chain 315.

The computing device 310 or other portion of the system 300 can include or interface with at least one controller 320. The controller 320 can include or interface with one or more processors and memory. The processor can be implemented as a specific purpose processor, an application specific integrated circuit (ASIC), one or more field programmable gate arrays (FPGAs), a group of processing components, or other suitable electronic processing components. The processors and memory can be implemented using one or more devices, such as devices in a client-server implementation. The memory can include one or more devices (e.g., random access memory (RAM), read-only memory (ROM), flash memory, hard disk storage) for storing data and computer code for completing the various operations described herein. The memory can be or include volatile memory or non-volatile memory and can include database components, object code components, script components, or any other type of information structure for supporting the various activities and information structures of the present disclosure. The memory can be communicably connected to any number of processors and include computer code or instruction modules for executing one or more processes described herein. The memory can include various circuits, software engines, and/or modules that cause the processor to execute the systems and methods described herein.

The controller 320 can include or be coupled with communications electronics. The communications electronics can conduct wired and/or wireless communications. For example, the communications electronics can include one or more wired (e.g., Ethernet, PCIe, or AXI) or wireless transceivers (e.g., a Wi-Fi transceiver, a Bluetooth transceiver, an NFC transceiver, or a cellular transceiver). The communications electronics can couple the controller 320 to one or more elements of the system 300, or various components of the controller 320 to each other. For example, the controller 320 can receive various data from the ultrasound probe 100 and generate data maps for presentation by a user interface (e.g., VB mode data or M mode data for manual review, or derivative data generated from such data maps). The controller 320 can cause one or more operations disclosed herein, such as by employing another element of the system 300. For example, operations disclosed by other elements of the system 300 can be initiated, scheduled, triggered, or otherwise controlled by the controller 320, via generation of control signals.

The computing device 310 can include a user interface to include the depicted display 330, or an audible, haptic or other input or output components configured to exchange information with a user or a further computing device. For example, the user interface can present a graphical depiction of a data map which may include or omit spatial information. Such data maps can include M-mode data maps generated from PE scans or VB-mode data maps generated from further QUS scans. The controller 320 can derive the various parameters corresponding to each scan from such data maps. In some embodiments, the data maps themselves, instead of or in addition to information derived therefrom, may be ingested into a model to characterize or predict a progression of a condition.

The display of the computing device 310 can present information, such as a patient identifier 311 (e.g., a personally identifiable or anonymized token corresponding to a subject). The display can present a prompt to initiate data gathering (e.g., a receipt of PE or QUS scan data, or an indication to save received data). The computing device 310 can further store any displayed or other information accessible thereto. For example, the computing device 310 can store the patient ID 311.

Referring now to FIG. 4A, a flowchart for a method 400 of characterizing an anatomical structure is provided, according to an illustrative example of the present disclosure. According to various embodiments of the present disclosure, various operations can be omitted, substituted, added or modified. For example, in some embodiments, the method 400 can omit the PE or other QUS scan, or employ further data received from various sources (e.g., age, intraocular pressure, sphere-equivalent refractive error, or inter-ocular pressure (IOP)).

At operation 405, PE scan data is received by the controller 320 (e.g., one or more processors thereof). The PE scan data can include a return signal from various ambient sources. At operation 410, QUS scan data (e.g., a signal indicative of BSC and envelope data) is received.

The PE scan data can be validated at operation 415. For example, the controller 320 can compare a number of stationary lines to a predetermined threshold, and validate the data responsive to a determination that the number of stationary lines exceeds the threshold. In some embodiments, operation 415 can be performed simultaneously or alternatively with operation 405 such that the controller 320 can cause an output of an indication of the validation of the data (e.g., an output to a user to adjust an orientation of the ultrasonic probe 100). At operation 420, the controller 320 can validate the BSC or envelope data of operation 410. For example, the controller 320 can compare a number of distinct RF echo lines to a predetermined threshold. In some embodiments, operation 420 can be performed simultaneously or alternatively with operation 410, such that the controller 320 can cause an output of an indication of the validation of the data (e.g., an output to a user to adjust an orientation of the ultrasonic probe 100), or automatically engage a trigger based on such validation.

At operation 425, the controller 320 can generate one or more parameters from the validated subset of the PE scan data (e.g., all PE scan data in an embodiment omitting operation 415). For example, the parameters can include a shear-wave speed, such as a maximum, average, standard deviation, or other aspect thereof. The parameters can include an axial displacement (e.g., length of the eye 200). At operation 430, the controller can generate one or more parameters from the validated subset of the QUS scan data (e.g., all QUS scan data in an embodiment omitting operation 420). The parameters can include a backscatter coefficient (BSC), and various envelope parameters such as an effective acoustic concentration (EAC), effective scatter diameter (ESD), various values of a Homodyned-K distribution (α parameter, μ parameters or so forth). As indicated above, in some embodiments, additional parameters can be captured from any source. For example, such information can include an anterior chamber distance, age, visual acuity, or so forth.

At operation 435, an anatomical structure can be characterized based on the various parameters. For example, the characterization of the structure can include a mechanical feature such as an elasticity or other mechanical property or microstructure of one or more loci of the eye 200. Such loci can include loci distributed along a depth of the eye 200, of along the surface of the eye 200. In some embodiments, the method can be performed longitudinally (e.g., monthly, annually, or so forth). The longitudinal change of parameters over time can form further parameters (e.g., A SWS, A EAC, A Age, A Visual Accuity, A Elasticity, A Microstructure Parameter). The longitudinal data can characterize a progression of a state of the eye 200.

Referring now to FIG. 4B, a flowchart for a method 450 of predicting a progression of a condition of an eye is provided, according to an illustrative example of the present disclosure. At operation 455, parameters, including any parameters disclosed herein (e.g., the parameters of the method 400 of FIG. 4A) are received for a first patient, and a plurality of further patients of interest (e.g., patients of a same or similar age, visual acuity, all available data, or other subpopulations thereof).

At operation 460, the method 450 includes ingesting, into a machine learning algorithm, the parameters. The machine learning model can include, for example, a classification model such as a random forest, k-nearest neighbors, regression based, or other model. In some embodiments, the machine learning model can include a binary classifier for one or more progressive conditions of an eye. Such conditions can include, for example, myopia, glaucoma, age-related macular degeneration, code-rod dystrophy, or other conditions. In some embodiments, the machine learning model can include a binary classifier (e.g., a support vector machine (SVM)). In some embodiments, the machine learning model can be substituted or employed in conjunction with regressive or other predictive models.

At operation 465, the binary or other classifier can classify a condition as associated (e.g., clustered) with progression of a condition greater than a threshold. Such a threshold can include a diopter threshold (e.g., corresponding to moderate myopia, high myopia, or pathological myopia). Such a threshold can include a structural threshold, such as a threshold related to a depth (e.g., thickness) of a sclera, an elasticity of one or more portions of the sclera, a change thereof, a dimension or location of a microstructure, or so forth. In some embodiments, the classifier or other model can predict a response to a treatment option. For example, a response to atropine eye drops can be predicted based on a clustering or other associated with a known response in the plurality of plurality of further patients. For example, a binary or other classifier can classify a response as effective or ineffective, or another machine learning model (e.g., explainable AI or a convolutional neural network) can predict a rate or degree of progression of a condition (e.g., myopia). In some embodiments, the method can further include a provision of the atropine eye drops to a patient, to treat the myopia, or another treatment particular to a medical condition (e.g., the myopia). Such a provision of the eyedrops or other treatment can be responsive to the predicted response to the treatment.

### EXAMPLES

The present technology is further illustrated by the following Examples, which should not be construed as limiting in any way.

### B. Clinical Example of Characterization of the Anatomical Structures of the Eye.

Myopia occurs when light entering the eye is focused anterior to the retina and is typically the result of excessive axial eye length. Progression of the disease to high myopia (HM), defined as spherical equivalent refractive error (RE)≤−5 diopters (D), results in significantly higher risk of progression to pathologic myopia (PM), a leading cause of blindness worldwide. The prevalence of myopia is increasing and becoming epidemic; current trends indicate half the global population will be myopic by 2050, with approximately 10% (1 billion) exhibiting HM. Although minor visual dysfunction resulting from low to moderate myopia can be treated with corrective lenses and eyeglasses, HM patients are at increased risk of PM. Up to 70% of patients with HM suffer from sight-threatening pathologies. PM often presents as myopic maculopathy or posterior staphyloma that can lead to irreversible vision loss.

The sclera is the main load-bearing tissue in the eye and is a primary contributor in maintaining eye shape. Changes in eye shape or axial length must be facilitated by anatomical and microstructural changes within the sclera. Studies in mammalian models have revealed the scleral collagen changes underlying myopia progression include scleral thinning, increased collagen degradation concurrent with decreased collagen synthesis, a decrease in collagen crosslinking, and thinner collagen fibril diameter. Uniaxial tension measurements of sclera tissue strips revealed the viscoelastic sclera is mechanically weaker and more compliant in myopic eyes. Previous investigations have shown that increased axial length in humans is associated with a thinner sclera in both the posterior segment and anteriorly near the scleral spur, but neither a history of increasing axial length nor in myopic refractive error, on its own, can effectively predict future myopia progression. Although age, ethnicity, and higher levels of myopia are indicators for future myopic progression, no sole demographic feature or current ophthalmic measurement can predict the rate at which myopia will develop in severity or identify patients are at imminent risk of PM. Direct, quantitative measurements of the biomechanical and microstructural changes of the sclera in vivo may be more predictive of myopia progression and identify patients at imminent risk of PM.

Imaging modalities for in vivo assessment of ocular microstructure and biomechanics have predominantly been applied to the cornea. Confocal microscopy is limited to the anterior eye but offers cellular-level resolution of the corneal layers, providing clinical information for disease detection and monitoring or evaluating treatment outcomes (e.g., refractive surgery). Conversely, optical coherence tomography (OCT) is capable of imaging ocular structures in both the anterior and posterior segments with micron-level resolution. Biomechanical properties of the cornea can be inferred by combining temporal OCT imaging with a force stimulus to induce tissue motion. For example, shear-wave propagation incited by an acoustic source or air puff can be tracked with OCT to estimate shear modulus by measuring the shear-wave speed. Passive elasticity imaging (i.e., without an external stimulus) of the cornea can be achieved by exploiting intraocular pressure fluctuations resulting from cardiac activity that cause compressive forces on the cornea which can be measured to compute local strains.

Optical techniques for imaging into and through the sclera are difficult because the dense, tortuous collagen fiber network renders the sclera optically opaque. Nevertheless, OCT has been used to visualize scleral layers and microvasculature at the corneo-scleral limbus and measure the anterior sclera thickness. Investigators have also applied elasticity imaging methods to estimate tissue stiffness using OCT and an external shaker to measure surface wave propagation along the sclera. Compared to OCT, Ultrasound (US) imaging is lower resolution but can more easily penetrate the sclera without significant signal attenuation. Due to the increased imaging depth, US elastography methods have been used in vivo to evaluate the mechanical properties of the optic nerve using shear-wave techniques or the posterior sclera-retina-choroid complex by analyzing pre- and post-deformation images during quasi-static loading. However, the long focal length of US transducers (1-2 cm) at typical clinical ophthalmic imaging frequencies (10-20 MHz) make elasticity imaging of the anterior globe difficult due to the need for acoustic coupling through water or US gel. Alternatively, anterior scleral stiffness can be estimated using Schiotz tonometry, although separating scleral stiffness from intraocular pressure in the measurements is difficult. To date, no quantitative imaging methods have been developed to parameterize the anterior sclera microstructure and biomechanical properties in vivo.

We have developed and described a novel high-frequency point-of-care (POC) ultrasound instrument to quantify the microstructural and biomechanical properties of the anterior sclera in vivo. The POC instrument is low-cost, easy-to-use, safe, and portable, enabling rapid scanning of patients during a clinical visit in addition to standard ophthalmic measurements. Because the anterior sclera is directly accessible and ~0.5 mm thick, the POC instrument utilizes an 80 MHz transducer with a short focal length (≈2.4 mm), small focal spot size (≈200 μm), and theoretical axial and lateral resolutions of 20 μm and 40 μm, respectively, that is suited for scanning the sclera stroma without reduction in signal quality due to attenuation from intervening tissue layers. Recent evidence showed that the microstructural properties of the anterior sclera are affected by myopia, indicating a relevance for high-frequency US due to superficial access and limited penetration depth of the US signal.

According to the present disclosure, radiofrequency (RF) echo data collected with the POC instrument are analyzed with quantitative ultrasound (QUS) methods to infer the biomechanical and microstructural properties of the anterior sclera. We have established QUS methods based on measurements of the backscatter coefficient (BSC) and envelope statistics as effective tools for quantitative soft tissue characterization. Several clinical and theoretical studies have demonstrated the efficacy of these QUS methods in many tissues and organs. For a target tissue, the transducer frequency is chosen so that the wave number k of the associated center frequency relates to the scatterer structure diameter D via the relationship k D≈2 to obtain a clinically useful sensitivity. For the 80 MHz transducer in the POC instrument, the preceding relationship implies an example of a clinically useful sensitivity to scatterers with D≈6 μm. Therefore, the resulting QUS parameters will be sensitive to the microstructural properties of collagen fibers comprising the sclera. In order to probe the biomechanical properties of the anterior sclera, a novel passive elastography (PE) algorithm is employed to quantify shear-wave propagation speed. Shear-wave speed (SWS) offers a parameter to characterize myopia-induced changes in the elastic properties of the sclera. Our previous studies demonstrated the sensitivity of QUS parameters to microstructural changes in the posterior sclera of a mammalian animal model caused by myopia and chemical crosslinking. Another study revealed correlations between the acoustic and biomechanical properties of the posterior sclera and its collagen microstructure. The set of QUS parameters comprising BSC, envelope statistics, and PE provide novel contrast mechanisms to characterize the biomechanical and microstructural properties of the anterior sclera and detect myopia-induced alterations. QUS parameters computed from data collected with our POC instrument of the anterior sclera relate to myopia level and can provide information to predict myopia progression and risk of PM.

In the cross-sectional study presented here, both eyes of patients with varying levels of myopia were scanned with our POC instrument. We describe the scanning protocol, review the QUS processing methods, describe an algorithm to automate data review and processing, and elucidate correlations between QUS parameters and myopia level. Current clinical assessments are limited to measurements that describe the existing level of myopia without directly characterizing the sclera microstructure or biomechanical properties. Unlike existing imaging modalities, such as angiography (fluorescein or indocyanine green) that permits study of vascularity or optical coherence tomography to observe tissue morphology, our non-invasive POC instrument and QUS processing methods provide unique insight into the properties of the sclera stroma at the micron scale. We relate the QUS parameters to measurements of RE and axial length to identify correlations between parametric values and level of myopia. This example relies on measurements at a single time point and thus does not determine longitudinal changes in QUS parameter values compared to myopia progression; however, the present example can be repeated to determine longitudinal relationships, such as relationships between myopia and QUS parameters characterizing the anterior sclera.

### 2. Methods

### 2.1. The High-frequency Point-of-Care Ultrasound Instrument

A detailed description of the example POC instrument, including design principles and acoustic output measurements, is provided. Various embodiments may add, remove, substitute, or omit features or levels of operation, according to various embodiments. The instrument is comprised of an 80 MHz transducer housed in a 3D-printed “stylus”; a desktop PC equipped with a digitizer card having a 500 MHz sampling frequency, 12-bit precision, and 400 mV reference voltage; a pulse generator to excite the transducer with a 1 ns impulse; a custom trigger circuit to synchronize transmission and reception at a pulse-repetition frequency (PRF) of 8070 Hz; and in-line filters and a pre-amplifier in the receive line for RF signal conditioning prior to digitization.

FIG. 5A shows a photograph of the “stylus” portion of the POC instrument. The 80 MHz transducer (FIGS. 5B-5C) attached to the stylus had a 1.2 mm aperture diameter and 2.4 mm focal length and is identical to the transducer used in previous studies characterizing the microstructural and biomechanical properties of the posterior sclera. The −6 dB bandwidth of the transducer was measured to be 48-120 MHz. Silicone caps (also referred to as end-effectors without limiting effect) to place over the transducer, as shown in FIG. 5D, were cast using a custom mold. These caps 1) protect the eye from the metal casing of the transducer and 2) provide the appropriate offset to ensure the transducer focus is located within the sclera stroma during data acquisition. A photograph of a volunteer being scanned with the POC instrument is shown in FIG. 5E.

### 2.2. Calculation of QUS Parameters

Table 1 provides a summary of all QUS parameters explicitly considered in this example. Two parameters are associated with the backscatter coefficient (BSC), two come from the Homodyned-K (HK) probability density function fit, and three are derived from the depth-dependent shear-wave speed (SWS) estimated from the passive elastography (PE) method. The remainder of this section describes how these parameters are calculated.

TABLE 1
Summary of QUS and PE parameters.

| QUS Method | Parameter | Tissue Property |
|---|---|---|
| BSC - GS Model | Effective Scatterer Diameter (ESD) | Scatterer (e.g., scleral collagen) |
| | Effective Acoustic Concentration (EAC) | diameter |
| | | Scatterer number density and |
| | | relative acoustic impedance |
| Envelope Statistics - HK | Scaling Factor (μ) Scatterer Clustering | Average magnitude of echo |
| | Parameter (α) | envelope |
| | | Number of scatterers per |
| | | resolution cell |
| Passive Elastography | Mean Shear-wave Speed (Mean SWS) | Mean shear modulus through |
| | Maximum SWS (Max SWS) | depth |
| | Standard Deviation of SWS (Std. SWS) | Maximum shear modulus |
| | Variation of shear modulus through depth | through depth |

RF data collected with the POC instrument were processed using QUS methods to compute parameters relating to the microstructural and biomechanical properties of the sclera. Parameters computed using methods based on measurements of the BSC and envelope statistics provide insight into tissue microstructure in terms of scatterer size, concentration, and spatial organization.

In the context of the sclera, the dominant scatterers are likely to include collagen fibers. To estimate the biomechanical properties of soft tissues, we employed the PE method to compute SWS from local tissue displacements, a parameter related to the tissue shear modulus. The QUS methods and corresponding parameters (Table 1) applied in this study have been described and validated in detail in previous publications. We briefly summarize the QUS methods involved in this study to provide context for the scanning protocol and interpretation of the results.

Acoustic scatterer size, number density, spatial organization, acoustic impedance, and shape affect the amplitude and frequency dependence of the backscattered RF echo spectrum. Using a reference phantom method, system effects are removed from the measured echo spectrum to obtain the frequency-dependent BSC. Tissue microstructure is parameterized by adopting and fitting an appropriate scattering model to the BSC curve. Our previous studies applying QUS methods to the posterior sclera (and other tissue types) implemented the Gaussian scattering model of the form

$$B S {C}_{G S} ( f ) = \frac{n {γ}_{0}^{2} {π}^{4} {f}^{4} {{V}_{s} ( {a}_{e f f} )}^{2}}{4 {π}^{2} {c}^{4}} exp ( \frac{- 0.8274 {π}^{2} {f}^{2} {a}_{e f f}^{2}}{{c}^{2}} ) ,$$

where n is the number density of scatterers, γ0 is the relative impedance mismatch per particle, a_{eff} is the effective scatterer radius, c is the speed of sound, and

$${V}_{s} ( {a}_{e f f} ) = \frac{4}{3} π {a}_{e f f}^{3}$$

is the scatterer volume. The effective scatterer diameter (ESD=2a_{eff}, in μm) and effective acoustic concentration

$$( EAC = 10 log ( n {γ}_{0}^{2} ) ,$$

in dB/m³) are estimated from this model and relate to tissue microstructure.

Envelope statistics-based methods estimate the probability density function that fits the histogram of RF echo envelope values. As with the scattering model fit to the BSC curve, QUS parameters of the fit probability density function relate to tissue microstructure. For this study, the Homodyned-K (HK) distribution was selected because the associated parameters provide insight into scatterer number density and spatial organization. The probability density function for the HK distribution is defined as

$$f H K ( r ) = r {\int }_{0}^{∞} u {J}_{0 ( u ∈ )} {J}_{0} ( u r ) {( 1 + \frac{{u}^{2} {σ}^{2}}{2 α} )}^{- α} \cdot du$$

In Eq. 2, J₀ is the 0th-order Bessel function of the first kind, σ² is the diffuse signal energy, and ε² is the coherent signal energy. The parameter α corresponds to the number of scatterers per resolution cell. A derived parameter k=ε/σ is the ratio of the coherent to diffuse signal energy. An additional parameter μ is computed from the mean intensity of the signal envelope and normalized by k to compute the mean diffuse signal intensity 67. A robust algorithm based on the first moment of intensity and two log-moments was recently developed to estimate parameters from the HK distribution even though it lacks a closed-form expression.

Parameters from the BSC and envelope statistics are computed from tissue signals within a region-of-interest (ROI), which is defined to be sufficiently large to reduce variance in the QUS parameters, small enough to span a homogeneous tissue region, and provide satisfactory spatial resolution. Thus, QUS parameters can describe the “average” tissue properties throughout the ROI even though the parameters relate to structures smaller than the ≈20 μm acoustic wavelength of the 80 MHz transducer. Estimating parameters from BSC and envelope statistics-based methods can include collecting a sufficient number of distinct (e.g., independent or uncorrelated) RF echo lines and is accomplished with the POC instrument by translating the stylus back and forth over a span of ≈1 mm across the sclera. The rate of RF line acquisition is set to a constant PRF and therefore may not be synchronized with motion of the stylus, meaning the POC instrument may not form images because no spatial information may be contained between sequential RF lines. Nevertheless, data are still sufficient for QUS parameter estimation. Biomechanical properties of the sclera can be inferred by measuring the propagation speed of shear-waves traveling through the tissue. Assuming the sclera exhibits linear-elastic material properties, the SWS is related to the tissue shear modulus and mass density. We implemented the PE method based on autocorrelation of noise-like displacement fields because, unlike active elasticity imaging methods that include an external stimulus to generate shear-waves, PE relies on tracking noise-like shear-waves generated by physiologic sources such as skeletal muscle or cardiac activity. Thus, the POC instrument can operation with no additional instrumentation to produce shear-waves.

Tissue motion for SWS estimation is tracked by recording RF data from the same tissue location over a span of time. This is accomplished by holding the POC stylus stationary at a single location on the sclera. The acquisition rate (i.e., PRF determined by the trigger circuit) and number of RF lines needed to be acquired are determined by the expected tissue shear modulus and resulting frequencies of propagating shear-waves. We determined that a PRF of at least 6.6 kHz and 150 RF lines can be employed to apply the PE method to data captured from the sclera stroma.

The output of the PE algorithm is a depth-dependent SWS curve that was parameterized by computing the mean (Mean SWS), maximum (Max SWS), and standard deviation of the SWS curve (Std. SWS) as shown in Table 1. Considering the axial extent of the sclera within the transducer focal zone, Mean SWS corresponds to the average tissue shear modulus, Max SWS is the largest observed shear modulus, and Std. SWS captures the variation of the sclera stiffness.

### 2.3. Study Design and US Data Collection

All subjects were recruited at the Singapore Eye Research Institute (SERI). This study was reviewed and approved by the Institutional Review Board of SERI and Weill Cornell Medicine. Informed consent was obtained from each patient. All research adhered to the tenets of the Declaration of Helsinki and the Health Insurance Portability and Accountability Act.

85 subjects (54 female, mean age 57.2±9.4 years) were recruited for this study. Each subject underwent standard ophthalmic measurements to obtain spherical equivalent RE (Canon RK5, Canon Inc Ltd, Tokyo, Japan); axial length (Axl, in mm); anterior segment biometric parameters such as anterior chamber depth (ACD, in mm), central corneal thickness (CCT, in μm), and total corneal power (K, in D); and intraocular pressure (IOP, in mmHg, Nidek NT-2000, Nidek Co Ltd, Hiroishi Gamagori, Aichi, Japan). Axl, ACD, CCT, and K were all measured using an IOLMaster 700 (Carl Zeiss Meditec, Inc., Dublin, CA). All subjects but one were phakic. The singular pseudophakic subject underwent cataract surgery more than five years before recruitment in this study.

RF echo data were collected from the temporal and nasal regions of the anterior sclera from both eyes of each subject. Prior to RF data collection, topical proparacaine 0.5% was instilled in each eye to achieve anesthesia. A sterilized silicone cap (10-minute soak in chlorhexidine gluconate (0.5% w/v in denatured alcohol 70% v/v) followed by 10-minute soak in sterile water) was filled with US coupling gel and subsequently placed on the transducer. The tip of the silicone cap was then positioned in contact with the anterior sclera region centered at approximately 5 mm posterior to the limbus along the horizontal meridian with the eye looking away (cardinal temporal gaze for nasal region scan, cardinal nasal gaze for temporal region scan). After ensuring the sclera stroma was in the focal zone of the transducer, data were collected as illustrated in FIGS. 6A-6D.

First, for a “PE Scan”, the transducer was held stationary to collect RF data from the same location on the sclera. These data were used for the PE algorithm to estimate SWS. Next, a “Virtual B-mode” (VB-mode) was acquired by sliding the transducer back and forth across the sclera surface over a range of ≈1 mm. A VB-mode has an appearance similar to a typical clinical B-mode image; however, the translation of the transducer was not synchronized to the PRF. Therefore, no lateral spatial information is encoded in the resulting RF echo frame.

Data were collected continuously once acquisition was initiated using a foot pedal. RF echo data were stored as frames of 1000 lines. Each frame represented ≈125 ms of data. Memory transfer latency reduced the effective frame-rate to ≈4 Hz. Up to 100 frames were stored in memory, providing the clinician sufficient time to complete both PE scans and VB-mode collection of the nasal or temporal region of an eye in the approximately 25 seconds before the image queue was filled. One investigator reviewed the collected data in real-time to check data quality. In some cases, the same eye and region was rescanned if the data either did not provide a sufficient number of stationary RF lines for PE processing or did not provide enough independent RF lines for BSC processing.

A representative PE scan and VB-mode are displayed in FIGS. 6A and 6B, respectively. Total scan time for each patient (i.e., both eyes and two locations per eye) was approximately 2-3 minutes. In the few cases where scanning took longer, the time increase was due to patient difficulty in keeping their eye open once the transducer was placed in contact with the sclera.

### 2.4 Semi-Automated QUS Processing

Scans with the POC instrument at each region of an eye can generate up to 100 frames containing 1000 RF lines. For the preliminary QUS results presented in, all acquired echo frames were manually reviewed and processed. To avoid this time-intensive process to identify regions for QUS processing and reduce operator-dependence, we developed a software to semi-automatically extract high-quality RF data to compile datasets for QUS processing. Details of the algorithm are provided in Sec. S1. The software was written in MATLAB (2022a, The Mathworks, Natick, MA).

For each eye region, the algorithm compiled RF lines into “BSC datasets” and “PE datasets” (Sec. S1). RF data compiled into a single BSC dataset per eye region were used to compute BSC and enveloped statistics-based parameters. Conversely, contiguous sets of RF lines compiled into one or more PE datasets for each eye region were used in the PE algorithm to estimate SWS. ROIs for both types of datasets were centered at the transducer focus of 2.3 mm and spanned 12 wavelengths (i.e., 200 μm) in the axial extent. Preliminary measurements showed that a minimum of 75 independent RF echo lines were necessary to achieve consistent BSC and envelope statistics-based parameter estimates. Any BSC datasets—from the temporal or nasal region—of an eye containing fewer than 75 lines of data were excluded from further processing. Similarly, contiguous blocks of at least 150 RF lines were used for computing SWS with the PE algorithm. PE datasets with fewer than 150 lines of data were excluded from further processing.

### 2.5 Statistical Analysis and Classification

All statistical analyses and classification were performed using Python 3.9 with the scikit-learn 1.3.0 and SciPy 1.10.1 libraries.

Eyes were subdivided into myopia groups based on refractive error as control (C, RE>−0.25), low/moderate myopia (LMM, −5.00≤RE≤−0.25), or high myopia (HM, RE<−5.0). Eyes diagnosed with a staphyloma were grouped as pathologic myopia (PM). QUS parameters were averaged over the temporal and nasal regions for each eye to generate a single parameter set.

Eyes were included only if PE and BSC datasets from both the nasal and temporal regions met the inclusion criteria based on dataset size as specified in Sec. 2.4.

We refer to the group of parameters Axl, ACD, CCT, K, IOP, and RE as “ophthalmic parameters” for brevity. Pearson correlation was computed to compare patient age, QUS, and ophthalmic parameters with RE, Axl, and age. Multi-linear regression was then performed to predict RE, Axl, or patient age. The independent variables for the multi-linear regression were the QUS parameters from eyes in the C, LMM, and HM groups only. PM eyes were excluded from the regression calculations because the presence of a staphyloma was expected to introduce large variance and potential errors when assessing for associations with the measurements of RE and Axl (e.g., the Axl as measured along the visual axis does not capture the full extent of the posterior protrusion when the staphyloma apex is off-axis, especially in cases where all staphyloma ridges are outside the visual axis). A one-way ANOVA was performed to compare the QUS and ophthalmic parameters across all four myopia groups, followed by Tukey post-hoc test to compare the parameter values between paired groups.

Finally, a quadratic discriminant analysis (QDA) classifier was evaluated using leave-one-out validation for predicting myopia group using QUS or ophthalmic parameters. All four groups were included in this analysis. The QDA classifier used default hyperparameter values in the scikit-learn library. Classifiers were trained using the seven QUS parameters or four ophthalmic parameters (ACD, CCT, K, IOP) with or without patient age (four classifiers total). Performance of the trained classifiers was evaluated using receiver-operating characteristic (ROC) curves and reported as area under the ROC curve (AUC). An ROC curve and the resulting AUC were generated for each of the four classes by each classifier using a one-vs-rest approach. A summary ROC curve was generated for each classifier using micro-averaging to report a single AUC value for the classifier performance.

### 2.6. Intra-operator Repeatability Measurements

Our previous report demonstrated the repeatability of QUS parameters when data were reviewed and regions manually marked for QUS and PE processing. We repeated the analysis in this study using the semi-automated algorithm. Intra-operator repeatability was tested by having the same operator scan all four regions of one subject's eyes five times in succession. Semi-automated QUS processing proceeded in the same manner as described in Sec. 2.4.

### 3. Results

Of the 170 eyes scanned, 110 were included in the final processing. Three eyes were excluded due to unreliable refractive error measurement, five eyes were excluded because of unreliable anterior segment measurements, and since only one pathologic myopia eye did not have staphyloma (only myopic macular degeneration), this eye was also excluded. The other 51 were eyes excluded because the PE and/or BSC datasets for the nasal and/or temporal region did not meet the inclusion criteria based on dataset size (Sec. 2.4). Table 2 specifies the number of eyes in each myopia group for statistical analyses and classifier training.

FIGS. 7A-7B display representative PE and BSC datasets after semi-automated processing of a control eye. The orange box indicates the focal region of the transducer, corresponding to the axial range gate used for QUS processing region. FIG. 7C is an example of the depth-dependent SWS curve computed from the PE processing applied to the RF data in FIG. 7A. Similarly, plots in FIG. 7D show the curve fits for BSC and envelope-statistics based processing using the RF data from FIG. 7B.

TABLE 2
Number of eyes in each of the myopia groups and corresponding range of D.
110 eyes were included in the statistical analyses and classifier training.

| | | Low/Moderate Myopia | High Myopia | Pathologic Myopia |
|---|---|---|---|---|
| | Control (C) | (LMM) | (HM) | (PM) |
| Count | 20 | 24 | 42 | 24 |
| RE (D) range | −0.25 to 4.13 | −4.63 to −0.38 | −14.75 to −5.00 | −22.38 to −7.00 |

Pearson correlation values between the QUS parameters or ophthalmic parameters and refractive error, Axl, and patient age are compiled in Table 3. Three of the QUS parameters—EAC, σ, and μ—were negatively correlated with refractive error (p≤0.05 for all), suggesting the anterior sclera undergoes microstructural changes with higher levels of myopia. As expected, axial length exhibited a significant negative correlation with refractive error (R=−0.84, p<0.001). Both patient age and ACD were also moderately correlated with refractive error (R=0.41, p<0.001, R=−0.28, p=0.009, respectively).

Only EAC and μ were found to be correlated with Axl (R=0.24, p=0.03 for both). Conversely, all QUS parameters except ESD were found to be negatively correlated with patient age.

TABLE 3
Pearson correlation between QUS and ophthalmic parameters with
refractive error, axial length, and patient age. Underlined
values indicate statistical significance of p < 0.05.

| | Refractive Error | | Axial Length | | Age | |
|---|---|---|---|---|---|---|
| Parameter | R | p-value | R | p-value | R | p-value |
| ESD | −0.08 | 0.45 | −0.09 | 0.41 | 0.18 | 0.10 |
| EAC | −0.28 | 0.009 | 0.24 | 0.03 | −0.58 | <0.001 |
| α | −0.24 | 0.03 | 0.16 | 0.14 | −0.44 | <0.001 |
| μ | −0.28 | 0.009 | 0.24 | 0.03 | −0.57 | <0.001 |
| Mean SWS | −0.16 | 0.15 | 0.07 | 0.53 | −0.44 | <0.001 |
| Max SWS | −0.19 | 0.08 | 0.08 | 0.49 | −0.42 | <0.001 |
| Std. SWS | −0.21 | 0.05 | 0.14 | 0.21 | −0.37 | <0.001 |
| Age | 0.41 | <0.001 | −0.21 | 0.05 | — | — |
| Axl | −0.84 | <0.001 | — | — | — | — |
| ACD | −0.28 | 0.009 | 0.42 | <0.001 | −0.17 | 0.13 |
| CCT | −0.09 | 0.40 | −0.002 | 0.98 | −0.12 | 0.26 |
| K | 0.07 | 0.48 | −0.41 | <0.001 | 0.02 | 0.84 |
| IOP | 0.03 | 0.75 | 0.01 | 0.93 | −0.07 | 0.51 |

A multi-linear regression comparing the set of QUS parameters to refractive error, axial length, or patient age showed statistically significant correlation for all (FIGS. 8A-8C). The correlation was moderate for refractive error (R=0.38, p<0.001) and axial length (R=0.35, p=0.001). The strongest correlation was found between the set of QUS parameters and patient age (R=0.66, p<0.001), consistent with the results of the individual Pearson correlation analyses.

Mean QUS and ophthalmic parameter values for the four myopia groups are tabulated in Table 4. The p-value from one-way ANOVA comparing the parameter values across groups are included in the right-most column (RE is excluded because it was used to separate the eyes into the C, LMM, and HM groups, Sec. 2.5). FIGS. 9A-9C display boxplots of ESD, EAC, and Mean SWS across the four groups.

Of the QUS parameters, EAC, u, Max SWS, and Std. SWS showed statistically significant differences when compared with ANOVA (Table 4). Interestingly, EAC (FIG. 9B), α, and μ, increased with level of myopia (decreasing refractive error) but decreased for PM cases. Similarly, Mean SWS, Max SWS, and Std. SWS increased from C to LMM, but decreased from LMM to HM and PM.

As expected because of its large correlation with refractive error, Axl was statistically significantly different between the groups (p=0.001). Patient age (p=0.001) and ACD (p=0.02) were also significantly different among the groups.

Pair-wise comparisons using Tukey post-hoc test (Table 5) revealed some differences in QUS and ophthalmic parameter values between myopia groups. Comparing C and HM eyes, both EAC (p=0.04) and μ (p=0.006) were significantly different. Std. SWS exhibited statistically significant differences between LMM and PM (p=0.01) and HM and PM (p=0.008). Mean difference in Max SWS between LMM and PM or HM and PM was nearly significant (p=0.05 for both). For the ophthalmic parameters, Axl differed pair-wise between all group (p≤0.01 for all) whereas ACD only reach significance between C and PM (p=0.02) and patient age differed between C and HM (p=0.001).

ROC curves generated by the four QDA classifiers are displayed in FIG. 10 and the AUC values are compiled in Table 6. Considering the overall classification performance based on the micro-averaged ROC curves and the associated AUC values, a QDA classifier trained with the QUS parameters (excluding patient age) most accurately differentiates the eyes (FIG. 10C, AUC=0.68). Interestingly, including patient age did not significantly change the micro-averaged AUC (FIG. 10D, AUC=0.67, p=0.37) and may suggest that incorporating additional input parameters does not necessarily improve classifier performance.

TABLE 4
Mean QUS and ophthalmic parameter values by myopia group. The
p-values were computed from one-way ANOVA across the groups.

| | Mean ± Stan. Dev. | | | | ANOVA |
|---|---|---|---|---|---|
| Parameter | C | LMM | HM | PM | p-value |
| ESD (μm) | 7.65 ± 0.20 | 7.65 ± 0.28 | 7.65 ± 0.29 | 7.79 ± 0.26 | 0.21 |
| EAC (dB/m³) | 151.5 ± 0.5 | 151.8 ± 0.9 | 152.0 ± 0.8 | 151.6 ± 0.5 | 0.03 |
| α | 0.16 ± 0.18 | 0.18 ± 0.22 | 0.27 ± 0.17 | 0.25 ± 0.15 | 0.08 |
| μ (×10⁻⁴) | 2.0 ± 4.0 | 2.4 ± 7.7 | 2.5 ± 6.1 | 2.3 ± 4.0 | 0.007 |
| Mean SWS | 0.84 ± 0.14 | 0.93 ± 0.16 | 0.92 ± 0.19 | 0.85 ± 0.15 | 0.11 |
| (m/s) | | | | | |
| Max SWS (m/s) | 1.43 ± 0.28 | 1.62 ± 0.30 | 1.60 ± 0.32 | 1.41 ± 0.21 | 0.01 |
| Std. SWS (m/s) | 0.26 ± 0.06 | 0.31 ± 0.07 | 0.30 ± 0.08 | 0.24 ± 0.05 | 0.002 |
| Age (years) | 61.6 ± 6.5 | 56.7 ± 10.1 | 52.9 ± 8.2 | 57.8 ± 8.2 | 0.001 |
| Axl (mm) | 23.5 ± 0.77 | 24.6 ± 1.05 | 26.7 ± 1.07 | 28.8 ± 1.70 | <0.001 |
| ACD (mm) | 3.06 ± 0.31 | 3.17 ± 0.25 | 3.26 ± 0.34 | 3.35 ± 0.31 | 0.02 |
| CCT (μm) | 515.0 ± 124.4 | 556.0 ± 36.9 | 549.7 ± 32.3 | 557.8 ± 36.8 | 0.09 |
| K (D) | 44.3 ± 1.42 | 44.2 ± 1.5 | 44.1 ± 1.31 | 43.8 ± 1.64 | 0.74 |
| IOP (mmHg) | 13.4 ± 2.98 | 14.7 ± 4.08 | 14.0 ± 2.61 | 14.8 ± 2.60 | 0.36 |

For classifying eyes by level of myopia, the QDA classifiers accepting ophthalmic parameters as input were better at identifying control eyes (AUC=0.72, including patient age) and LMM eyes (AUC=0.68, excluding patient age). Conversely, QDA classifiers performed better at identifying HM eyes (AUC=0.62, including patient age) and PM eyes (AUC=0.71, excluding patient age) when trained using the QUS parameters. Notably, when comparing the classification performance of each class, all four classifiers performed the worst at identifying HM eyes.

TABLE 5
Paired Tukey post-hoc tests comparing QUS and ophthalmic
parameter values between myopia groups. Underlined
values have statistical significance of p < 0.05.

| | C | | | LMM | | HM |
|---|---|---|---|---|---|---|
| Parameter | LMM | HM | PM | HM | PM | PM |
| ESD | 0.99 | 1.0 | 0.37 | 0.95 | 0.51 | 0.18 |
| EAC | 0.38 | 0.04 | 0.97 | 0.79 | 0.63 | 0.11 |
| α | 0.98 | 0.13 | 0.34 | 0.26 | 0.55 | 0.99 |
| μ | 0.06 | 0.006 | 0.46 | 0.95 | 0.67 | 0.26 |
| Mean SWS | 0.24 | 0.28 | 1.0 | 0.99 | 0.32 | 0.38 |
| Max SWS | 0.13 | 0.14 | 0.99 | 0.99 | 0.05 | 0.05 |
| Std. SWS | 0.13 | 0.14 | 0.86 | 0.99 | 0.01 | 0.008 |
| Age | 0.18 | 0.001 | 0.38 | 0.29 | 0.97 | 0.11 |
| Axl | 0.01 | <0.001 | <0.001 | <0.001 | <0.001 | <0.001 |
| ACD | 0.70 | 0.11 | 0.02 | 0.66 | 0.17 | 0.64 |
| CCT | 0.13 | 0.17 | 0.10 | 0.98 | 1.0 | 0.96 |
| K | 1.00 | 0.97 | 0.72 | 1.0 | 0.84 | 0.87 |
| IOP | 0.50 | 0.89 | 0.41 | 0.80 | 1.0 | 0.70 |

TABLE 6
AUC values for quadratic discriminant analysis (QDA) classifier.

| | QUS Parameters | | Ophthalmic Parameters | |
|---|---|---|---|---|
| Myopia | Excluding | Including | Excluding | Including |
| Group | Age | Age | Age | Age |
| C | 0.68 | 0.71 | 0.66 | 0.72 |
| LMM | 0.66 | 0.62 | 0.68 | 0.65 |
| HM | 0.60 | 0.62 | 0.58 | 0.55 |
| PM | 0.71 | 0.67 | 0.60 | 0.57 |
| Averaged | 0.68 | 0.67 | 0.64 | 0.65 |
| AUC | | | | |

Underlined values indicate highest AUC for each group.

Results of the repeatability study are shown in Table 7. The mean±standard deviation of each QUS parameter for each eye are included. Results are consistent with our previous finding where ESD and EAC had low variability while the envelope statistics parameters and SWS exhibited larger variation.

TABLE 7
Mean ± standard deviation of QUS parameters computed from five repeated measurements
of the same volunteer's eyes. Underlined parameters exhibit standard deviation <15% of the mean.

| | ESD | EAC | | | Mean SWS | Max SWS | Std. SWS |
|---|---|---|---|---|---|---|---|
| Eye | (μm) | (dB/m3) | α | μ | (m/s) | (m/s) | (m/s) |
| OD | 5.45 ± 0.28 | 93.7 ± 1.12 | 2.19 ± 1.17 | 0.003 ± 0.001 | 1.14 ± 0.31 | 1.80 ± 0.50 | 0.32 ± 0.13 |
| OS | 5.21 ± 0.32 | 93.2 ± 0.45 | 1.42 ± 0.40 | 0.002 ± 0.0003 | 0.84 ± 0.19 | 1.44 ± 0.37 | 0.28 ± 0.10 |

### 4. Discussion

The high-frequency POC instrument enables US data acquisition from the anterior sclera of patients in vivo. QUS methods operating on RF echo data to compute parameters relating to the biomechanical and microstructural properties of the sclera do not require full image formation, simplifying the required instrumentation and in vivo data acquisition procedures. QUS parameters correlated with refractive error and, when used as input features for a QDA classifier, achieve satisfactory performance at determining the current level of myopia within an eye, suggesting the QUS parameters may be sensitive to tissue property changes within the anterior sclera. Scanning both eyes of a cohort of patients with varying levels of myopia, and a subgroup exhibiting pathologic myopia in the form of staphyloma, provided the foundation for investigating the relationship between myopia and QUS parameters associated with the anterior sclera.

The current study served to establish the feasibility of our QUS methods to evaluate the anterior sclera. Correlations between the quantitative parameters and myopia severity suggests the QUS parameters are sensitive to myopia-induced biomechanical and microstructural changes within the anterior sclera. Three of the QUS parameters—EAC (R=−0.28, p=0.009), α (R=−0.24, p=0.03), and μ (R=−0.28, p=0.009)—were found to negatively correlate with RE. Both EAC and α are related to the number density of scatterers and may imply a reduction in collagen fibers with myopic progression, consistent with previous findings of general scleral collagen degradation in myopic eyes. A multi-linear regression of all seven QUS parameters revealed correlations with RE (R=0.38, p<0.001), Axl (R=0.35, p<0.001), and age (R=0.66, p<0.001). Age affects scleral stiffness and leads to microstructural changes in collagen fibrils, including an increase in the sclera collagen fibril diameter and intermolecular lateral spacing. All QUS parameters except scatterer diameter (ESD) correlated with age, indicating the parameters are sensitive to age-related changes in the scleral biomechanical and microstructural properties. The relatively weaker correlation between QUS parameters and RE and Axl may suggest the QUS parameters provide complementary information to standard ophthalmic measurements with limited redundancy. That is, a high correlation in the multi-linear regression would imply the QUS parameters provide no additional information beyond what is available from RE and Axl for assessing myopia. A lack of correlation would mean QUS parameters provide no information related to microstructural changes associated with myopia. The observed correlation is encouraging evidence that parameters computed from our QUS methods can detect microstructural and biomechanical changes in the anterior sclera and potentially provide biomarkers for predicting myopia progression and more accurate risk assessment for staphyloma formation beyond the information gleaned from current ophthalmic measurements.

When comparing the values of QUS parameters across groups via one-way ANOVA, EAC, μ, and Max SWS exhibited statistically significant differences. The pair-wise comparison among myopia groups (Table 5) revealed discrepancies occurred between C and HM eyes (EAC and μ), LMM and PM eyes (Std. SWS), or HM and PM eyes (Std. SWS). Values in Table 4 indicate that EAC and μ increase with greater levels of myopia (more negative refractive error) but decrease in the presence of a staphyloma. Similarly, Std. SWS increased from C to LMM, then decreased for HM and PM. These results may indicate a longitudinal change in the QUS parameters. Indeed, all QUS parameters except for ESD express a negative correlation with patient age, but the rate at which this change occurs may vary based on the level of myopia. These changes may occur prior to, concurrent with, or following changes in refractive error. There is potential for exploiting QUS to predict changes in myopia (e.g., where detected tissue alterations precede increases in myopic progression). If the QUS parameters change prior to staphyloma formation, measurements with our POC instrument could provide critical clinical information for assessing imminent pathologic myopia risks. The performance of the QDA classifiers provides further evidence that the QUS parameters are detecting myopia-induced biomechanical and microstructural changes in the sclera that complement standard ophthalmic measurements. Results shown in FIG. 10 and Table 6 indicate that, overall, QDA classifiers trained with only QUS parameters as input performed best at differentiating eyes based on level of myopia. When evaluating individual groups and excluding patient age, QDA with QUS was best able to classify C, HM, and PM cases whereas QDA with ophthalmic parameters better identified LMM eyes, albeit the AUC values were not statistically different (p≥0.55 for all). Age was included as input to the second set of classifiers because it is correlated with refractive error and microstructural properties of the eye are known to change with increasing age. However, for QDA trained with QUS parameters, incorporating age decreased performance overall and for LMM and PM eyes individually. Similar results were obtained for QDA trained with ophthalmic parameters. These results provide additional evidence that the QUS parameters capture information about biomechanical and microstructural changes in the anterior sclera that is complementary to standard ophthalmic measurements.

Shear-wave speed is estimated pixel-wise in the PE algorithm used in this study. As a result, the SWS parameter is depth-dependent through the transducer focal zone within the sclera stroma. Evidence exists that the stiffness of the episclera differs from the stroma and that stroma stiffness is depth-dependent. OCT elastography methods estimate sclera stiffness through the entire tissue thickness and therefore cannot detect depth-dependent changes. Computing the maximum and standard deviation of SWS captures some of the depth-dependent variation that may occur with age and myopia. Interestingly, Table 3 shows a negative correlation between Mean, Max, and Std. SWS and age, the opposite of what has been reported from tensile measurements, although an unexpected decrease in corneal stiffness with age has been observed based on shear-wave OCT elastography. The opposing findings may be a consequence of comparing stroma stiffness (as measured from the PE method and our POC instrument) to measurements via whole-globe dilation or strip extensiometry. Additional study into depth-dependent stiffness changes may buttress these findings. Nevertheless, SWS as estimated by the PE algorithm, especially the depth-dependence variation as captured by Std. SWS, differed among the four myopia groups and may be employed to predict myopia progression.

Implementing the semi-automated QUS processing algorithm may realize real-time analysis. In the present study, 51 eyes were excluded from statistical analysis caused by insufficient data in the PE and BSC datasets. The semi-automated processing algorithm currently returns conservative estimates of sclera surface location and correlation between RF lines in PE or BSC datasets to ensure high-quality data for processing. More sophisticated filtering and artifact detection, can reduce the number of excluded eyes resulting from small PE and BSC dataset sizes. Furthermore, incorporating fully automated QUS processing into the POC instrument will provide real-time feedback during scans to indicate when sufficient, high-quality data have been acquired for analysis.

Utilization of improved semi-automated QUS processing algorithms may result in higher quality data to further investigate the relationship between QUS parameters from the anterior sclera and myopia. More importantly, longitudinal data can include, among some subjects, progression in their level of myopia and possible staphyloma formation. The longitudinal data can be employed to predict myopia progression and better assess risk of staphyloma formation according to the systems and methods described herein.

### Supplementary Material

### S1. Semi-Automated QUS Processing

Scans from a single region of the anterior sclera can generate a large volume of data. Many of the echo frames are not used for QUS processing because of incorrect targeting (i.e., transducer focus not in sclera stroma) or significant axial motion resulting from movement of the patient's eye or operator's hand. As part of our goal toward real-time imaging, we have developed a semi-automated algorithm to compile RF echo lines into datasets for further QUS processing.

The algorithm first identified all RF lines in each 1000-line frame where the transducer focus was located within the sclera stroma. Given the focal distance of ≈2.4 mm, this was accomplished by detecting the location of the sclera surface within each RF line via cross-correlation with a reference signal (FIG. 11A). The magnitude of the curve resulting from the cross-correlation was then median filtered to remove the effects of spurious correlations due to noise. FIG. 11B displays an example of the cross-correlation before and after applying the median filter. The sclera surface was defined as the location of the first point in the filtered cross-correlation with a magnitude ≥0.008 (green horizontal line in FIG. 11B), a value determined empirically. Once the sclera surface location was found for all RF lines in a frame, a Hampel filter was applied to suppress spurious misidentifications most often caused by noise. Only RF lines where the detected surface was at a distance of 2±0.04 mm from the transducer, indicated by the vertical blue lines in FIG. 11B, were retained for further processing.

After detecting all RF echo lines with the sclera surface within the target distance, the mean of the RF signal envelope was computed for each line in the 2.4-2.5 mm axial range. All signals with a mean envelope value less than 0.03 V (determined empirically) in the specified range gate were discarded because of insufficient signal-to-noise ratio. Finally, selected RF lines were retained only if they occurred in contiguous groups of at least two. Again, this was implemented to avoid including data where the detected sclera surface was incorrectly found to be within the target axial range.

For SWS estimation, the PE algorithm uses a contiguous block of RF signals exhibiting low global motion. The algorithm found regions in single echo frames where at least 150 consecutive RF lines had the sclera surface at the target distance. Then, the zero-lag correlation was computed between the signal envelope of all lines in the data block over the 2.15-2.45 mm axial range. The envelope was used because phase differences are expected from shear-wave propagation, but the envelope is mostly affected by global motion (e.g., transducer movement). If more than one usable block of data was found in an echo frame, the block containing the largest number of contiguous RF lines was saved for processing. We refer to these as “PE datasets”. Multiple PE datasets could exist for each eye region scanned.

The algorithm operated similarly for identifying RF data suitable for BSC and envelope statistics-based processing. However, unlike some PE datasets, the independent RF echo lines may not be in a contiguous block. Estimating the BSC from backscattered echo data uses independent, uncorrelated RF lines. Assuming the microstructural properties of the sclera are homogeneous over the region captured in the VB-mode, any independent RF echo line can be included in QUS processing. Therefore, after identifying all RF lines within an echo frame where the detected sclera surface was in the target region, the zero-lag correlation between RF signals was computed over the 2.15-2.45 mm axial range. Any lines with correlation ≥0.95 (determined empirically) were excluded. The correlation was computed pair-wise between all RF lines. Any pair with correlation larger than the specified threshold were removed. After processing each echo frame, the set of uncorrelated RF lines were compiled into a single, larger echo frame for BSC and envelope-statistics based processing. We refer to these as “BSC datasets”. A single BSC dataset was created for each eye region scanned.

BSC and PE datasets were manually reviewed prior to QUS processing. Regions were removed that exhibited reflection artifacts, had the sclera surface mis-identified, or demonstrated significant signal loss. Example of PE and BSC datasets before and after manual identification of poor quality data regions are shown in FIGS. 12A-12F. The PE dataset displayed in FIG. 12A contains regions of global displacement caused by movement of the transducer or eye. The region manually identified in FIG. 12B displays little to no global motion. Extracting region 1 results in a high-quality dataset for PE processing shown in FIG. 12C. Similarly, an automatically generated BSC dataset (FIG. 12D) contains regions of RF lines that should be excluded, identified as “2” and “3” in the red boxes in FIG. 12E. RF lines in region 2 had significant signal loss in the focal region. RF lines in region 3 also exhibited an apparent reduction in SNR and a large “speckle” size, suggesting high correlation between the RF lines that may indicate exclusion for BSC-based processing. After removing regions 2 and 3, the remaining BSC data set shown in FIG. 12F remains.

### C. Another Clinical Example of Characterization of the Anatomical Structures of the Eye.

### Introduction

High myopia, or extreme nearsightedness, is a leading cause of blindness worldwide, especially when it progresses to pathologic myopia. Currently, no method exists to assess myopia development and severity quantitatively in vivo. Myopia is typically evaluated with measurements of axial length and refractive error. While these measures are useful to provide guidance for vision correction and are correlated with increased risk of pathologic myopia, they provide no direct information about the progression of pathologic changes within the eye tissues. Nor can these measures alone predict the likelihood of vision-threatening staphyloma formation in the posterior region of the eye.

In myopia, images are focused anterior to, rather than upon, the retina. More than 95% of myopia cases result from excessive axial length in the eye. Globally, myopia affects up to 2.3 billion people, and the prevalence is rising throughout the world. Although minimal levels of myopia are considered a minor inconvenience, high pathologic myopia can lead to irreversible vision loss. The average axial length of the adult emmetropic eye is approximately 24 mm. High myopia is defined as an axial length >26 mm or <−6.0 dioptres. The prevalence of high myopia has been increasing and is now considered epidemic in some populations. Not only does the sclera of these extremely elongated, highly myopic eyes show marked thinning, but some thin to the extent that local outpouchings (staphyloma) form. Curtin reported a 34.5% prevalence of blindness among 403 staphylomatous eyes. Recent reports note that a higher grade of staphyloma is associated with further eye elongation and more severe pathologic myopia-related maculopathy, which is the leading cause of blindness in Japan and the second leading cause in China.

The anatomical changes underlying eye elongation and staphyloma formation are likely to occur within the sclera. In mammalian models, scleral thinning and tissue loss occur during the development of myopia. Decreased scleral collagen synthesis, increased collagen degradation, decreased collagen-fibril diameter and decreased collagen crosslinking have been reported in mammalian models and high myopic patients. The sclera is a viscoelastic structure and becomes more flexible and less load-bearing in myopes than in normal eyes. Moreover, there is little evidence to suggest that scleral changes in myopia are limited to the posterior pole. The thickness of the sclera in a non-myopic human eye varies with location from ~1 mm near the optic nerve, 0.9 mm in the posterior pole, 0.3 mm under the insertion of the rectus muscles (anterior to the equator) and then increases slightly to 0.5 mm in the anterior sclera. Studies have shown that increased axial length in humans is associated with a thinner sclera both posteriorly and anteriorly near the scleral spur. In addition, human sclera shows increasing stiffness with age in a highly region-dependent manner, with a more pronounced increase in stiffness in the anterior sclera. With age, scleral changes include increased collagen crosslinking and glycosylation, decreased type-1 collagen, increased collagen fibre diameter and variability and decreased decorin, biglycan, scleral hydration and elastin, which collectively result in increased scleral stiffness.

In this paper, we describe the development and testing of a novel point-of-care (POC) high-frequency (i.e., ≈80-MHz center frequency) ultrasound (US) instrument to assess myopia quantitatively, in vivo, as manifested by microstructural and biomechanical changes in the anterior sclera. The high frequency corresponds to the thickness of the human anterior sclera (i.e., ~0.5 mm) because the POC instrument acquires high-quality echo data from within the stroma at adequate spatial resolution. Quantitative ultrasound (QUS) methods will operate on data acquired with the POC device to assess the microstructural and biomechanical properties of the anterior sclera in human subjects in vivo. QUS analyses raw, radiofrequency (RF) US echo signals to infer quantitative properties of tissues. The premise underlying QUS-based tissue-characterization studies is that these properties (i.e., QUS parameters) are different for normal and myopic sclera and can provide information about disease progression. QUS metrics include envelope statistics and the backscatter coefficient (BSC).

Quantitative ultrasound methods based on the BSC and envelope statistics are sensitive to scattering from structures much smaller than can be resolved on conventional B-mode US. Based on wavenumber k=2πF_{c}/c for center frequency F_{c} and speed of sound c, these QUS methods best characterize scatterers of size D such that kD≈2. This yields D≈6 μm at 80 MHz. Backscattered signals are principally determined by the scatterer size when this criterion is satisfied. Rayleigh scattering occurs when kD<<2, whereas other features, including scatterer shape, begin to affect the backscattered echo signal when kD>2. Therefore, BSC and envelope statistics-based QUS parameters computed from 80 MHz echo data are sensitive to changes occurring in the collagen during myopia progression, providing a new unique quantitative contrast mechanism in the anterior sclera.

An additional QUS parameter of interest is the tissue shear modulus to summarize biomechanical properties of the sclera. For this, the innovative passive elastography (PE) method is a QUS technique that will also operate on data acquired by the POC device. PE exploits tissue motion induced by physiologic sources (e.g., pulsating blood vessels) to estimate shear-wave speed in the anterior sclera that is related to shear modulus. Shear-wave speed provides another QUS parameter to quantify changes in elastic properties likely to occur in the myopic sclera. Shear-wave speed combined with parameters computed from the BSC and envelope statistics provides a novel contrast mechanism to assess changes in the scleral stroma.

This manuscript focuses on the POC device that received Institutional Review Board approval at the Singapore Eye Research Institute for a human study. The POC device is low-cost, easy-to-use, portable and safe. Therefore, the POC device could become a standard instrument used during yearly eye examinations to assess the risk of developing vision-threatening pathologic myopia. Currently, clinical assessment is limited to instruments that ascertain the degree of existing myopia, but no method exists to quantify scleral biomechanical properties or to predict progression to more vision-threatening stages. As discussed in our recent review article, existing imaging modalities such as angiography (fluorescein or indocyanine green) is confined to studying vascularity, and optical coherence tomography provides only limited anatomic information about the sclera because of limited penetration. Neither method provides the biomechanical properties that our novel POC device would provide.

### Methods

The POC device can collect RF echo data from the anterior sclera that can be processed with our QUS algorithms. Unlike typical ultrasonography applications that form two-dimensional (2D) images, the POC device uses a single handheld transducer that rapidly collects one-dimensional data (e.g., it does not form images of spatial information). In the remainder of this section, we describe the design of the POC device, strategies for repeatable data acquisition and how quantitative information will be derived from RF echo data.

### System Requirements

To acquire high-frequency US data effectively and safely from the anterior sclera for quantitative analysis, the POC device can include: a handheld transducer to allow for robust data acquisition; a user interface to provide real-time feedback and save data (e.g., anonymized data); compliance with safety and health data regulations and recorded echo data having sufficiently high signal-to-noise ratio (SNR) and repeatability for quantitative analysis.

Creating a handheld device can aid the operator to acquire data easily from different regions of the anterior sclera. Coupled with a simple user interface to display real-time feedback and save anonymized data, a single user can operate the device with no additional assistance. Compliance with health and safety regulations constrains the acoustic pressures that can be generated by the high-frequency transducer, thus reducing the achievable SNR. The POC device can apply a transmission pressure within regulatory constraints to provide repeatable estimates of QUS parameters with low variance.

### Transducer

The POC device includes a transducer. The centre frequency and bandwidth are selected as high enough to ensure sensitivity to tissue microstructural properties at the target scale (i.e., ≈6 μm) but sufficiently low to avoid excessive acoustic attenuation and permit the US beam to penetrate into the stroma of the anterior sclera. Our group has used a high-frequency transducer (≈80 MHz) with an axial resolution of ≈20 μm to characterize the microstructure of the posterior sclera in myopic guinea pig models and normal pig eyes, which are ≈200 μm and ≈600 μm thick, respectively. RF echo data obtained in those studies exhibited sufficiently high SNR and penetration depth of the US beam. For these reasons, an identical high-frequency transducer was chosen for the POC device. The single element lithium niobate transducer is spherically focused with a 1.2 mm aperture diameter and 2.4 mm focal length. FIG. 13 displays photographs of the transducer.

With an outer diameter of 8.2 mm and short focal length, the transducer is small enough to allow scanning of the anterior sclera with relative ease (e.g., no open shell system as for US biomicroscopy). A biocompatible parylene layer is deposited on the transducer, but an additional layer of protection can be used to ensure the transducer does not harm the patient's eye. Furthermore, given the focal distance and tight focal spot, the transducer can be held ≈2 mm away from the eye to ensure the focal spot is targeted within the scleral stroma. We created custom silicone caps to protect the eye and aid the user in orienting the transducer. These caps shield the eye from direct contact with the transducer housing while providing the appropriate offset to align the sclera within the focal zone.

### System Block Diagram And Components

The remaining POC system components were designed around the transducer to optimize the signal and collect data suitable for QUS processing. A block diagram for the proposed POC device is shown in FIG. 14. The primary components are the handheld transducer, a computer to provide the user interface and house the analog-to-digital converter, a foot switch to start and stop data acquisition, a pulse generator to excite the transducer, a trigger source to control pulse excitations and synchronize data recording and filters and preamplifier in the receive chain for signal conditioning prior to digitization.

Apart from the transducer, the user interface and foot switch are the only system components that the user interacts with. The interface provides real-time feedback during data acquisition, aids the user to review collected data and saves the anonymized data. Real-time feedback can help guide the operator during the scanning process and collect data suitable for QUS analyses. Once the scan is complete, the ability to review collected data aids the user to select specific acquisitions for QUS processing and discard any unwanted data. Selected datasets can then be saved securely (e.g., with anonymized information for later post-processing).

Considerations of the analog-to-digital converter include the sampling rate, bit precision and reference voltage. Temporal sampling requirements are guided by the Nyquist criterion of at least twice the largest frequency component of the recorded signal, although a rule of thumb is 10× the center frequency to increase SNR. Similarly, the bit precision and reference voltage determine the discrete signal amplitudes that can be recorded. Higher bit precision reduces digitization noise. In some embodiments, the reference voltage is near the maximum expected amplitude to ensure full bit usage. However, there is a cost trade-off: higher sampling rate and bit precision can significantly increase analog-to-digital converter cost, and high-speed cards typically have a fixed reference voltage (i.e., not adjustable). The analog-to-digital converter selected for the POC device has sufficient sampling rate, bit precision and voltage reference so that echo data have the SNR necessary for QUS processing.

The trigger source sets the pulse repetition frequency (PRF) of the POC device. Acquisitions are enabled and disabled with the foot switch, which enables and disables the trigger, but the trigger source controls the rate at which data are acquired. The PRF is an important parameter for PE. Specifically, the PRF determines the maximum shear wave frequency that can be detected. The PRF was chosen to cover the expected shear wave bandwidth without increasing the pressure field intensity beyond limits set by health and safety regulations (see section below on safety regulations). Previous studies using PE to assess the mechanical properties of the posterior sclera suggest a PRF of 8 kHz is adequate.

The pulser and signal conditioning electronics in the receive chain have an effect on the recorded echo data. The transmitted US pulse should have a wide bandwidth (short pulse duration) and sufficient pressure amplitude to achieve the SNR necessary for QUS processing. To achieve this, the pulser can generate a short e.g., (≤2 ns), high-voltage electrical signal to excite the transducer. The amplitude of the electrical excitation signal can be selected to keep the emitted pressure below a threshold (e.g., limits set by health and safety regulations for certain in vivo subjects (see section below on safety regulations)). An additional attenuation component is included at the pulser output to optionally decrease the excitation voltage. In the receive chain indicated in FIG. 14, the preamplifier boosts the echo signal to an amplitude range compatible with the analog-to-digital converter, whereas the filters can remove frequency components of the signal outside the pulse bandwidth.

Another component is the handheld transducer. This pen-like piece provides a means for the user to comfortably hold and position the otherwise small high-frequency transducer. At the front end of the handheld component is a connector for the transducer. A coupler at the back end allows electrical connection to the pulser and receive signal chain. The length of the handheld piece was partially based on the length of the coaxial cable connecting the transducer to the coupler. An appropriate cable length may aid in effective transmission and reception of the RF signal with minimal distortion. Care was taken to select the cable length to ensure a large amplitude, wideband US pulse. However, the lengths of coaxial cables connecting the coupler to the pulser, and receive chain and cables connecting other system components, had a small effect on the echo data.

### Safety Measurements

The United States Food and Drug Administration (FDA) marketing clearance of diagnostic US systems and transducers § 5.2.8 defines limits of the pressure field allowed for use in ophthalmic applications. The derated spatial-peak, temporal-average intensity cannot exceed 50 mW/cm² and the mechanical index cannot surpass 0.23. To ensure our POC device complies with FDA limits, the pressure field of the high-frequency transducer was measured using a 40 μm diameter needle hydrophone (Precision Acoustics, acoustics.co.uk). Both the hydrophone and its associated booster amplifier (Precision Acoustics, acoustics.co.uk) were calibrated by Physikalisch-Technische Bundesanstalt over the 0.1-100 MHz frequency range in increments of 0.1 MHz.

Exposimetry measurements of the pressure field were performed with the hydrophone submerged in a beaker of degassed, deionized water. The hydrophone was connected to the booster amplifier, and the output of the amplifier was digitized at a 1 GHz sampling rate at 8-bit resolution using a PXI-5152 oscilloscope (National Instruments, ni.com). The transducer was attached to a linear positioning system and manually aligned with the hydrophone to maximize the recorded pulse signal. Then, by raster-scanning the transducer in two dimensions with a step size of 2 μm, a 600×600 μm² RF volume was collected by the hydrophone. Given the approximate beam-width of 20 μm for the transducer, the step size and scan area ensured the peak pressure was recorded in the raster scan.

Following pressure field data collection, the RF signal containing the peak pressure amplitude was extracted and further processed to compute the derated intensity and mechanical index. Spatial averaging effects caused by the large size of the needle hydrophone element compared with the focal spot size of the transducer were corrected to rescale the measured pressure amplitudes p(t). The pulse intensity integral of the pressure wave was then calculated,

$$PII = \frac{1}{ρ C} {\int }_{0}^{T} {ρ}^{2} ( t ) d t$$

where c and ρ are the speed of sound and mass density of water, respectively. Derating of the pulse intensity integral was done by assuming tissue attenuation to be 0.3 dB/MHz/cm and that the pulse travels 200 μm from the sclera surface to the focus of the transducer. Derated intensity was then computed by multiplying the integral with the PRF of the trigger source. The mechanical index was computed using the derated maximum pressure amplitude pₘₐₓ and same center frequency used for derating the pulse intensity integral:

$$MI = {P}_{max} / \sqrt{{F}_{c}}$$

All levels accorded with regulatory limits. In an embodiment, the derated I_(spta.3) was 8.6 mW/cm\hat{ }2, and MI was ≥0.22.

### QUS Processing

Data collected with the POC device can be used to evaluate the microstructural and biomechanical properties of the anterior sclera. QUS methods based on measurements of the tissue BSC and envelope statistics have been applied at high frequency (80 MHz) to assess microstructural changes in the posterior sclera. Similarly, PE has been employed to measure shear modulus changes in the posterior sclera caused by chemical crosslinking. We briefly summarize the QUS and PE methods for clarity and to motivate the in vivo scanning protocol in the next section of the paper.

The amplitude and frequency dependence of the backscattered RF echo spectrum arises from the size, shape, number density, spatial organization and variations in acoustic impedance of scatterers within a medium. By removing system effects from the measured spectrum, the frequency-dependent BSC can be estimated and used to infer microstructural properties of biologic tissues. Quantitative parameters summarizing the tissue microstructure can be extracted by fitting the measured BSC to an appropriate scattering model. In our work, we employed a Gaussian scattering model, which assumes the scatterers exhibit continuous fluctuations in acoustic properties:

$$B S {C}_{G S} ( f ) = \frac{n {γ}_{0}^{2} {π}^{4} {f}^{4} {{V}_{s} ( {a}_{e f f} )}^{2}}{4 {π}^{2} {c}^{4}} exp ( \frac{- 0.8274 {π}^{2} {f}^{2} {a}_{e f f}^{2}}{{c}^{2}} ) ,$$

where n is the number density of scatterers, γ₀ is the relative impedance mismatch per particle, a_{eff} is the effective scatterer radius, c is the speed of sound and

$${V}_{s} ( {a}_{e f f} ) = \frac{3}{4} π {a}_{eff}^{3}$$

is the average particle volume. From this model, the effective scatterer diameter (2a_{eff}, in μm) and effective acoustic concentration (10 log(nγ₀²), in dB/m³) are estimated. Additional spectral parameters are obtained by fitting the measured BSC to a linear model to compute spectral slope (in dB MHz⁻¹ sr−1 m⁻¹), spectral intercept (in dB sr 1 m⁻¹) and midband fit (in dB sr⁻¹ m⁻¹). Although the linear spectral parameters do not directly define scattering properties of a medium, they are related to scatterer size and acoustic concentration. Parameters from both the Gaussian scattering model and linear model have been successfully used to quantify microstructural changes in the sclera.

Similarly, the histogram estimated from measured RF echo envelope values can be fitted to a probability density function. Parameters from the Nakagami distribution can be related to features of tissue microstructure, particularly the concentration of scatterers. This distribution takes the form

$${f}_{Nakagami} ( r ) = \frac{2 {m}^{m} {r}^{2 m - 1}}{Γ ( m ) {Ω}^{m}} exp ( - \frac{m}{Ω} {r}^{2} ) U ( r )$$

where Γ and U are the gamma function and step function, respectively. The parameters of interest are m, which corresponds to the number of scatterers per resolution cell, and Ω, which captures the average magnitude-squared of the envelope. Because the Nakagami probability density function is a closed-form expression, parameters can be estimated by fitting the function to an estimated histogram using a maximum-likelihood estimator.

Parameters from more complex models like the Homodyned-K (HK) distribution can offer additional insight into the scatterer number density and organization. The probability density function for the HK distribution is defined as

$${f}_{H K} ( r ) = r {\int }_{0}^{∞} u {J}_{0} ( u ε ) {J}_{0} ( u r ) {( 1 + \frac{{u}^{2} {σ}^{2}}{2 α} )}^{- α} d u$$

In Equation 5, J₀ is the 0th-order Bessel function of the first kind, σ² is the diffuse signal energy and 82 is the coherent signal energy. Like m from the Nakagami distribution, a corresponds to the number of scatterers per resolution cell. A derived parameter k=ε/σ is the ratio of the coherent to diffuse signal energy. Scatterers organized in a more structured or periodic fashion will increase the value of k, whereas scatterers more randomly distributed will reduce the parameter value. The lack of a closed-form expression for the HK distribution makes parameter estimation more difficult, though a robust algorithm based on the first moment of intensity and two log-moments exists. We found that parameters from the Nakagami and HK distributions were correlated with microstructural changes in the posterior sclera associated with myopia and chemical crosslinking.

Both BSC-based and envelope statistics parameters describe the ‘average’ tissue properties over a region-of-interest (ROI). ROIs are defined to be large enough to obtain consistent estimates of QUS parameters but small enough to provide adequate resolution. However, spatial resolution is less relevant for at least some embodiment of the POC device because no images are formed in at least some embodiments. Rather, typical ROI guidelines are useful to direct data collection from the anterior sclera. In this application, distinct RF echo lines (e.g., uncorrelated or independent RF echo lines) are collected to obtain data to compute QUS parameter values from both the measured BSC and envelope statistics.

Passive elastography is a technique to infer the mechanical properties of soft tissues by estimating the speed of propagating shear waves. Unlike active elasticity imaging methods that require an external stimulus to induce shear waves, PE relies on tracking tissue motion caused by noise-like shear waves created by physiologic sources (e.g., muscular or cardiac activity). PE has been applied in US applications, magnetic resonance imaging and optical coherence tomography. Shear-wave speed is estimated from measured time-dependent tissue motion. Because shear-wave speed in soft tissues is predominantly determined by the shear modulus, it can be used to infer tissue mechanical properties.

Tissue motion is tracked by processing motion-mode (M-mode) echo data collected from a given location over a span of time. Much like Doppler US, the transducer is maintained in a stationary position while echo data are recorded from the same tissue location. The PRF, which determines the rate at which echo lines are recorded, and the number of lines acquired are informed by the expected frequencies of propagating shear waves. Assuming the human sclera exhibits a shear modulus of ≈0.3 MPa and that the shear wavelength cannot be longer than the thickness of the anterior sclera to avoid conversion into a Lamb wave (≈500 μm as a conservative estimate), the range of expected shear wave frequencies spans 200 Hz-3.3 kHz. Thus, the PRF must be greater than 6.6 kHz, which means our proposed PRF of 8 kHz is sufficient.

To estimate shear-wave speed, a phase tracking algorithm first operates on the echo data to obtain pixel-wise displacement estimates. Then, a correlation-based algorithm is employed by the PE method to obtain shear wavelength and frequency, from which shear-wave speed is computed. A full description of the PE method has been previously published.

Table 8 provides a summary of 10 QUS parameters—five from the BSC, four from envelope statistics and the shear-wave speed—that will be computed from data collected with the POC device.

TABLE 8
Summary of QUS parameters estimated from radio frequency echo data.

| Method | Parameter | Microstructure feature | Scan protocol |
|---|---|---|---|
| BSC-GS model | Effective scatterer diameter | Scatterer diameter | VB-mode |
| | Effective acoustic | Scatterer number density and relative | VB-mode |
| | concentration | acoustic impedance | |
| BSC-Linear | Spectral slope | Scatterer diameter | VB-mode |
| model | Spectral intercept | Scatterer diameter, number density, | VB-mode |
| | | and relative acoustic impedance | |
| | Midband fit | Integrated backscatter intensity | VB-mode |
| Envelope | Scaling factor (Ω) | Average magnitude of echo envelope | VB-mode |
| statistics- | Shape parameter (m) | Number of scatterers per resolution | VB-mode |
| Nakagami | | cell | |
| Envelope | Structure parameter (κ) | Ratio of diffuse signal power | VB-mode |
| statistics-HK | Scatterer clustering | Number of scatterers per resolution | VB-mode |
| | parameter (α) | cell | |
| PE | Shear wave speed | Shear modulus | PE |

Abbreviations: BSC, backscatter coefficient; GS, Gaussian scattering; HK, Homodyned-K; PE, passive elastography; QUS, quantitative ultrasound; VB-mode, virtual B-mode.

### In Vivo Scanning Protocol

During a human subject investigation, the POC device can be used to collect RF echo data from the temporal and nasal regions of the anterior sclera. A scanning protocol was designed to record data for QUS processing. Just before beginning a scan, a silicone cap was placed on the transducer and an acoustic coupling medium added to the tip. Once the foot switch is actuated and the device begins acquiring echo data, the system continuously collects echo frames comprising 1000 RF lines at the 8 kHz PRF set by the trigger circuit. A queue of up to 100 frames is stored in memory. Then, during a scan, the transducer is oriented on the eye to ensure the sclera is within the focal zone (i.e., sclera surface ≈2 mm from transducer) by touching the cap to the sclera surface, followed by data collection in the two ways illustrated in FIG. 15. In the ‘PE Scan’, the transducer is held at one location to acquire M-mode acquisitions for shear-wave speed estimation. Next, for the ‘Virtual B-mode’ (VB-mode), the transducer is moved ≈1 mm along the sclera surface to record B-mode like data. Although this may not form an ‘image’, the translation is employed to collect independent echo data from a region of the sclera for estimating the BSC and envelope statistics-based parameters. Several frames will be recorded for both PE scans and VB-modes to ensure sufficient data are collected. The continuous data recording and extended image queue aid the performance of both scans in succession and within a few seconds.

Representative non-spatial ‘images’ (also referred to as data maps, without limiting effect) of the type of data collected during a PE and VB-mode scans are included at the bottom of FIG. 15. These data were collected from the eye of a volunteer. M-mode data recorded during a PE scan correspond, in some respects, to M-mode measurements currently used in clinical ultrasonography (e.g., echocardiography). In some embodiments, spatial information is encoded only within the axial range—the x-axis of the image corresponds to time and is determined by the PRF. Likewise, the spatial information encoded in echo data collected during a VB-mode can be limited to the axial dimension. The sweep of the transducer across the sclera is not necessarily synchronised with the PRF, meaning the lateral extent may be unknown. Although the resulting image may appear similar to a typical B-mode, it is important to note spatial information may not be available in the lateral dimension. A B-mode like appearance does, however, suggest independence of RF lines. Therefore, the PE scan can collect frames that have no apparent changes in the echo data, whereas a VB-mode can generate a B-mode like image.

### Results

We have successfully built and tested the POC device. The US field produced by the transducer has satisfactory bandwidth and SNR for quantitative analysis while meeting health and safety regulations for ophthalmic US. These results can aid the investigation of high-frequency QUS to detect microstructural changes within the anterior sclera in vivo that may be associated with the progression of myopia.

### Assembled System

Photographs of the fully assembled POC system are displayed in FIG. 16a. The user interface is a custom software solution developed using Lab View 2022 (National Instruments). During data acquisition, the user interface displays a real-time view of the 1000 RF line frames as they are acquired. The software also allows the user to view a queue of the recorded data, up to the last 100 frames as described in the section entitled in vivo scanning protocol, and select a range of frames to save. In addition to an anonymized patient identifier, the interface aids the user to specify eye and scan location for each set of acquisitions. This information is stored when the data are saved, facilitating post-processing and data analysis.

The photograph at the top right of FIG. 16a shows the custom designed, 3D-printed, handheld transducer assembly. Visible at the left and right ends are the high-frequency transducer and coupler, respectively. Partially contained within the apparatus is the coaxial cable connecting the transducer to the coupler. A 25 cm long cable conveyed the pulse transmitted to the transducer. A representative pulse obtained from a planar reflector and the associated frequency spectrum are shown in FIG. 16b, c, respectively. The −6 dB transducer bandwidth extends from 48 to 120 MHz, with a center frequency ≈85 MHz. We chose to make the handheld piece 16.5 cm long and introduce a loop in the cable to avoid bulk and make the device easier to handle. With a mass of 90 g, the handheld transducer is sufficiently ergonomic for the scans.

Silicone caps were cast using EcoFlex™ 00-30 silicone and custom 3D-printed molds. This silicone was chosen for its biocompatibility and flexibility. Two different geometries for the caps were created: one extended in a conical shape from the face of the transducer and the other in a cylindrical shape. Both cap types had a 10 mm outer diameter and a 3 mm opening in the center to avoid interfering with the acoustic field. The conical shape may facilitate scanning the sclera of patients whose eyelids cannot be opened wide enough to accommodate the diameter of the caps. On the contrary, the cylindrical cap shape makes maintaining the 2 mm offset easier. Both types of caps cover the face of the transducer and extend 4 mm up the sides, ensuring the transducer housing does not directly contact the eye.

The remaining electrical components are shown in the bottom-left photograph of FIG. 16a. The analog-to-digital converter is an ATS9350 digitizer card with 12-bit precision, 500 MHz sampling rate and variable reference voltage. The GZ1121GP-01 pulser creates a 400-V, 1-ns impulse that is attenuated by 23 dB using inline attenuators. In the receive chain are three in-line filters and a 50-dB low-noise pre-amplifier. The specific filters, amplifier and their configuration were chosen to condition the recorded signal: the RF echo first passes through a 41- to 800-MHz bandpass filter BHP-50+, is amplified AU-1480-BNC, and is further filtered by a 98-MHz low-pass filter BLP100+ and another 41- to 800-MHz bandpass filter. The trigger is a custom-built device using a 555 timer in a stable oscillator configuration. Because of tolerances with electrical components, the resulting PRF was measured to be 8.07 kHz.

### Safety Measurements Results

A photograph of the exposimetry set-up is displayed in FIG. 17a. The RF pulse with the maximum measured pressure as determined by the needle hydrophone is shown in FIG. 17b. To reduce the pressure of the pulse generated by the transducer and meet FDA guidelines, the output of the pulser was attenuated by 23 dB. Measured pressure and intensity were derated as specified in the section entitled safety measurements with F_{c}=85 MHz (FIG. 16c). With the 23 dB attenuation applied at the pulser output, the derated spatial-peak, temporal average intensity for the pulse was found to be 8.6 mW/cm² (FDA limit: ≤50 mW/cm²). Based on the peak pressure, the mechanical index was found to be 0.22 (FDA maximum limit: 0.23).

### In Vivo Test Data

In vivo measurements were made on one healthy volunteer to test the proposed scanning protocol and ensure collected data were applicable for quantitative analysis. Prior to data collection, two to three drops of proparacaine were applied to the right eye of the volunteer to anesthetize the eye and increase comfort during scanning. A silicone cap—sterilized by soaking in a solution of 70% ethanol for 10 min and then washed in phosphate-buffered saline—was placed on the transducer. GenTeal Tears™ eye drops were applied to the opening of the silicone cap to provide acoustic coupling. Then, the tip of the silicone cap was placed in contact with the nasal or temporal region of the anterior sclera. After ensuring the sclera was within the focal zone of the transducer, both VB-modes and PE scans were performed. The transducer was in contact with the volunteer's eye for ≈10 s during the scan of either the temporal or nasal region. FIG. 18 shows a photograph of the volunteer being scanned with the POC device and representative data collected in the PE scans and VB-modes.

### Illustrative QUS Results

Prior to QUS processing, the SNR of the in vivo RF echo data was evaluated. A VB-mode was selected and the mean spectrum computed over the 2.1- to 2.4-mm axial range gate, chosen because this range gate falls within the depth of field of the transducer. The SNR was computed as the difference between the spectral peak in the 48-120 MHz bandwidth—the −6 dB bandwidth of the transducer as shown in FIG. 16c—and the mean of the noise signal in the 200-250 MHz bandwidth. Echo data collected from the anterior sclera exhibited a SNR greater than 20 dB, making it suitable for further QUS processing. Representative VB-mode and PE scan data were then processed with our algorithms to evaluate the feasibility of these methods to process echo data captured with the POC device.

The left image in FIG. 19a displays the PE scan data chosen for further analysis. An ROI was selected that spans 2.1-2.4 mm in the axial range and across most of the RF lines in the 1000-line acquisition, as shown by the red box. A phase-tracking algorithm operated on the RF data to estimate tissue motion, the results of which are displayed in the middle image of FIG. 19a. Estimated tissue displacements were input to the PE algorithm to compute the depth-dependent shear-wave speed, as shown in the right plot of FIG. 19a. The mean shear-wave speed was found to be 0.69 m/s.

Similarly, a ROI was defined for the representative data for the VB-mode and is shown in the left image of FIG. 19b. The ROI again spans 2.1-2.4 mm in the axial range, but the lateral extent was chosen to ensure selected RF lines were uncorrelated. BSC-based QUS parameters were calculated by fitting the Gaussian scattering model to the measured BSC over the 48-120 MHz transducer bandwidth. The measured BSC and results of the scattering model fit are shown in middle plot of FIG. 19b. Effective scatterer diameter was found to be 8.03 μm, whereas the effective acoustic concentration was 148 dB/m³. Moreover, the red curve representing the Gaussian scattering model fit is in excellent agreement with the measured BSC, providing encouraging evidence that QUS parameters associated with the chosen model are applicable to describing the microstructural properties of the anterior sclera.

The histogram of the signal envelope in the same ROI was then estimated and the Nakagami and HK probability density functions were fitted, as shown in the right plot in FIG. 19b. Of the two distributions, the HK (red-dashed curve) provides a much better fit of the histogram than the Nakagami distribution.

Finally, a series of measurements were acquired to investigate the repeatability of data acquisitions with the POC device. The nasal region of a healthy volunteer's right eye was scanned five times. Contact between the transducer cap and the patient's eye was broken between each scan. One VB-mode and one PE frame were selected from each of the five scans to estimate all 10 QUS parameters. Table 2 contains the mean and standard deviations of the parameter estimates over all five scans. BSC-based parameters exhibited standard deviations <10% of the mean. For m of the HK distribution and shear-wave speed, the standard deviation was closer to 12% and 18%, respectively. Other parameters associated with the envelope statistics (Ω, κ and α) varied more significantly.

TABLE 9
Mean ± standard deviation of quantitative ultrasound (QUS) parameters computed
from five repeated measurements from the same region of a volunteer's eye.

| ESD | EAC | SS | I₀ | MBF | Ω | | | | SWS |
|---|---|---|---|---|---|---|---|---|---|
| (μm) | (dB/m³) | (dB/[sr m MHz]) | (dB/[sr m]) | (dB/[sr m]) | (log [V²]) | m | κ | α | (m/s) |
| 7.57 ± 0.20* | 153 ± 1.13* | 0.07 ± 0.008* | 32.8 ± 1.48* | 39.0 ± 1.14* | 0.003 ± 0.001 | 0.82 ± 0.10* | 0.10 ± 0.15 | 3.72 ± 2.66 | 1.24 ± 0.23 |

Note:
Parameter values marked by a “*” exhibit standard deviation <15% of the mean.
Abbreviations: EAC, effective acoustic concentration; ESD, effective scatterer diameter; I₀, spectral intercept; m, shape parameter; MBF, midband fit; SS, spectral slope; SWS; shear wave speed; α, scatterer clustering parameter; κ, structure parameter; Ω, scaling factor.

### Discussion

The POC device is operational and fully satisfies all design requirements. RF echo data collected by the device have sufficient SNR for QUS and PE processing. The inclusion of silicone caps on the handheld transducer makes scans of the anterior sclera straightforward and repeatable. Acquisitions are nearly instantaneous and both PE scans and VB-modes can be completed on the nasal or temporal region of the eye within a few seconds, reducing patient discomfort during the scanning process.

Targets for data acquisition with the POC device can include the nasal and temporal regions of the anterior sclera. Although the superior and inferior regions are also of interest, the size of the transducer complicates data collection from those sites. Other ophthalmic US devices permit scanning with the transducer in contact with the eyelid, which would ease access to the superior and inferior portions of the anterior sclera; however, the attenuation introduced by the eyelid would significantly degrade the SNR of the high-frequency transducer and complicate further quantitative processing. Increasing the excitation voltage to the transducer could boost the signal and increase the SNR, but the resulting derated intensity and/or mechanical index may surpass regulatory limits or other relevant threshold. A transducer design with a smaller outer diameter may be employed to improve data collection within all segments of the anterior sclera.

Echo frames are recorded as sets of 1000 RF lines to aid real-time display and provide a large enough window to collect sufficient data during a VB-mode or PE scan. It is not necessary for all RF lines in an echo frame to satisfy requirements for quantitative analysis. For example, we estimate that 100 adjacent RF lines ( 1/10 an echo frame, 12.5 ms acquisition time) can be used to estimate the shear-wave speed. QUS processing can use 10 or more distinct (e.g., independent) RF lines based on guidance for determining minimum ROI size in conventional QUS imaging. The RF lines do not have to be part of a contiguous block of data; that is, RF lines from different echo frames can be combined to compute parameters from the BSC and envelope statistics. Extending the acquisition window to 1000 lines and maintaining a queue of 100 frames aids sufficient data recordation within a few seconds of scan time for all QUS processing.

Data collected with the POC device can be manually reviewed and processed offline. Real-time QUS processing can be performed. Given the small dataset sizes, and because 2D parametric images were not formed, the QUS and PE algorithms operate fast enough to compute parameter values in seconds. For PE, echo data is complicated by global motion caused by transducer or eye movement. One approach to automatically detect a data block for PE processing is through correlation of the signal envelopes across the RF lines. Operating on the signal envelope discards phase information and, thus, ignores the micron-scale displacements caused by shear wave propagation. In this case, high correlation would indicate minimal global motion. Conversely, BSC and envelope statistics-based processing can use independent RF echo lines, which could also be determined based on the correlation among raw RF lines and identifying signals that are uncorrelated.

QUS parameters describe the average scattering properties over a small volume of tissue. Therefore, echo data for parameter estimation may be collected from portions of the sclera exhibiting the same scattering properties. Movement of the patient's eye and operator's hand will often increase the variance of parameter estimates. Table 2 is encouraging that the variance for in vivo scanning will be manageable.

The current design does not include dedicated circuitry for precise measurement of the relative orientations of the transducer and sclera. If the microstructural properties of the anterior sclera are indeed affected by myopia estimating those differences as a function of eccentricity from the anterior pole, the POC should incorporate further detection of such eccentricity (e.g., visual indications, pressure distributions, etc.). Further detection could also sense if the transducer is tilted relative to the scleral surface, to account for any distortion of the acoustic beam and increased variance of QUS parameters.

Some embodiments of the POC device could be made more portable and ergonomic. In particular, the weight and length of the handheld transducer could be reduced. At present, the 3D-printed housing is 165 mm long and has a mass of 90 g when assembled with the coaxial cable coupler and transducer. The length was a trade-off governed by the length of the coaxial cable and maintaining easy maneuverability. We chose to use a shorter length and introduce a loop in the co-axial cable. No hindrance to scanning was caused by the loop during the in vivo acquisition tests. A shorter coaxial cable, and thus a shorter and lighter handheld assembly, could be possible with a different transducer that better matches the 5002 impedance typical of some transmission lines. Alternatively, custom-made cables or an impedance matching circuit could improve the transmission lines between the transducer, pulser and receiving electronics.

The POC system is relatively low cost. Much of the expense is due to the analog-to-digital converter, high-frequency transducer and pulser. In all, expenses for all components in the current prototype system were less than US$15,000. Additional POC systems could be easily assembled and deployed at other institutions with minor financial hurdles. Finally, the system could be made more portable by replacing the desktop PC with a laptop and suitable analog-to-digital converter, as is typically done in current portable US systems.

### D. Example Systems, Methods, and Devices of the Present Disclosure.

Some sample embodiments are disclosed below, in order to represent illustrative embodiments, which one skilled in the art will understand may be further modified, combined, constrained, etc. according to the entirety of this disclosure. For example, any of the systems provided below can include any of the ultrasound probes, and any of the methods provided below can be performed with any of the systems or otherwise with the ultrasound probes.

Embodiment A: An ultrasound probe. The ultrasound probe includes a transducer to generate ultrasonic pressure waves at an acoustic frequency to penetrate the surface of the eye to a depth of an anatomical structure of the eye. The ultrasound probe includes a biocompatible end effector to contact a surface of the eye to propagate the ultrasonic pressure waves from the transducer to the eye. The ultrasound probe includes a sensor to detect a return pattern of the ultrasonic pressure waves, the return pattern indicative of a mechanical feature of the anatomical structure.

Embodiment B: Any of the embodiments disclosed herein (e.g., Embodiment A) wherein the biocompatible end effector is configured to directly contact the surface of the eye at a terminal end.

Embodiment C: Any of the embodiments disclosed herein (e.g., Embodiments A or B) wherein the biocompatible end effector includes a longitudinal dimension between the transducer and the terminal end equal to a distance to a focal spot corresponding to the transducer, less a distance from the anatomical structure to the surface of the eye. The biocompatible end effector includes a conformal material to conform to the surface of the eye. The biocompatible end effector can include an ultrasound transmission gel along the terminal end.

Embodiment D: Any of the embodiments disclosed herein (e.g., any of embodiments A to C) wherein the anatomical structure can be or include the anterior sclera.

Embodiment E: Any of the embodiments disclosed herein (e.g., any of embodiments A to D) wherein the acoustic frequency is in a range between 48 and 120 megahertz (MHz).

Embodiment F: A system. The system includes a transducer, a biocompatible end effector, and one or more processors. The transducer generates ultrasonic pressure waves at an acoustic frequency to penetrate the surface of the eye to a depth of an anatomical structure of the eye. The transducer detects a return pattern of the ultrasonic pressure waves, the return pattern indicative of a mechanical feature of the anatomical structure. The biocompatible end effector is configured to propagate the ultrasonic pressure waves between the transducer and the surface of the eye. The one or more processors are configured to analyze the return pattern to characterize the mechanical feature of the anatomical structure.

Embodiment G: Any of the embodiments disclosed herein (e.g., Embodiment F) wherein the anatomic structure is the anterior sclera. The mechanical feature includes an elasticity of the anterior sclera. The one or more processors are configured to, based on the characterization, predict a progression of myopia.

Embodiment H: Any of the embodiments disclosed herein (e.g., any of embodiments F or G) wherein the characterization is based on a plurality of parameters determined or reviewed by the one or more processors. The parameters include a sphere equivalent refractive error of the eye; an axial length of the eye; a maximum speed of a shear wave determined from the return pattern; a mean speed of the shear wave determined from the return pattern; a standard deviation of the shear wave speed determined from the return pattern; an effective scatterer diameter determined from the return pattern; an effective acoustic concentration determined from the return pattern; an anterior chamber depth of the eye; u parameter of a Homodyned-K (HK) distribution determined from the return pattern; a parameter of the HK distribution.

Embodiment I: Any of the embodiments disclosed herein (e.g., any of embodiments F to G) wherein one or more processors are configured to receive a plurality of longitudinal return patterns, the longitudinal return patterns corresponding to a longitudinal time of greater than one year. The one or more processors are configured to predict, based on the plurality of longitudinal return patterns and the longitudinal time, a future progression of the condition of the eye.

Embodiment J: Any of the embodiments disclosed herein (e.g., any of embodiments F to I) wherein the acoustic frequency is in a range between 48 and 120 megahertz (MHz).

Embodiment K: Any of the embodiments disclosed herein (e.g., any of embodiments F to J) wherein the one or more processors are configured to determine, based on the return pattern, that the transducer should be reoriented. The one or more processors are configured to provide, via a user interface, an indication that the transducer should be reoriented.

Embodiment L: Any of the embodiments disclosed herein (e.g., any of embodiments F to K) wherein the one or more processors are configured to compare the mechanical feature to a mechanical feature threshold and provide, via the user interface, an indication of the mechanical feature, based on the comparison.

Embodiment M: Any of the embodiments disclosed herein (e.g., any of embodiments F to L) wherein the mechanical feature comprises a plurality of elasticities corresponding to a plurality of loci of the anatomical structure.

Embodiment N: Any of the embodiments disclosed herein (e.g., any of embodiments F to M) wherein the wherein the one or more processors are configured to present an indication of the mechanical features corresponding to the plurality of loci.

Embodiment O: Any of the embodiments disclosed herein (e.g., any of embodiments F to N) wherein the one or more processors are configured to predict a progression of a condition of the eye and provide, via the user interface, the prediction

Embodiment P: Any of the embodiments disclosed herein (e.g., any of embodiments F to O) wherein the one or more processors are configured to, during a first mode of operation, cause the transducer to receive a first portion of the return signal from ambient sources. During a second mode of operation temporally offset from the first mode of operation, the one or more processors are configured to cause the transducer to generate the ultrasonic pressure waves and receive a second portion of the return signal.

Embodiment Q: Any of the embodiments disclosed herein (e.g., any of embodiments F to P) wherein the one or more processors are configured to generate a first portion of a data map from the first portion of the return signal, the first portion of the data map corresponding to a stationary position of the transducer relative to the eye. The one or more processors are further configured to generate a second portion of a data map from the second portion of the return signal, the second portion of the data map corresponding to a moving position of the transducer relative to the eye.

Embodiment R: A method. The method can include using the system of claim 1 to characterize a mechanical feature of an anatomical structure of the eye of a patient. The method can include causing the systems or methods above to perform their respective functions.

Embodiment S: Any of the embodiments disclosed herein (e.g., Embodiment R) wherein the characterization of the anatomical structure is predictive of a progression of myopia.

Embodiment T: Any of the embodiments disclosed herein (e.g., Embodiments R or S) wherein the subject is human.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. An ultrasound probe comprising:
a transducer to generate ultrasonic pressure waves at an acoustic frequency to penetrate a surface of an eye to a depth of an anatomical structure of the eye;
a biocompatible end effector to contact the surface of the eye to propagate the ultrasonic pressure waves from the transducer to the eye; and
a sensor to detect a return pattern of the ultrasonic pressure waves, the return pattern indicative of a mechanical feature of the anatomical structure.

2. The ultrasound probe of claim 1, wherein the biocompatible end effector is configured to directly contact the surface of the eye at a terminal end.

3. The ultrasound probe of claim 2, wherein the biocompatible end effector comprises:
a longitudinal dimension between the transducer and the terminal end equal to a distance to a focal spot corresponding to the transducer, less a distance from the anatomical structure to the surface of the eye;
a conformal material to conform to the surface of the eye; and
an ultrasound transmission gel along the terminal end.

4. The ultrasound probe of claim 1, wherein the acoustic frequency is in a range between 20 and 150 megahertz (MHz).

5. The ultrasound probe of claim 1, wherein the anatomical structure is an anterior sclera.

6. A system comprising:
a transducer to:
generate ultrasonic pressure waves at an acoustic frequency to penetrate a surface of an eye to a depth of an anatomical structure of the eye; and
detect a return pattern of the ultrasonic pressure waves, the return pattern indicative of a mechanical feature of the anatomical structure;
a biocompatible end effector to propagate the ultrasonic pressure waves between the transducer and the surface of the eye; and
one or more processors to:
analyze the return pattern to characterize the mechanical feature of the anatomical structure.

7. The system of claim 6, wherein:
the anatomic structure is an anterior sclera;
the mechanical feature comprises an elasticity of the anterior sclera; and
the one or more processors are configured to, based on the characterization, predict a progression of myopia.

8. The system of claim 6, wherein the characterization is based on a plurality of parameters determined or reviewed by the one or more processors, the parameters comprising a plurality of:
a sphere equivalent refractive error of the eye;
an axial length of the eye;
a maximum speed of a shear wave determined from the return pattern;
a mean speed of the shear wave determined from the return pattern;
a standard deviation of the shear wave speed determined from the return pattern;
an effective scatterer diameter determined from the return pattern;
an effective acoustic concentration determined from the return pattern;
an anterior chamber depth of the eye;
μ parameter of a Homodyned-K (HK) distribution determined from the return pattern; or
α parameter of the HK distribution.

9. The system of claim 6, wherein the one or more processors are configured to:
receive a plurality of longitudinal return patterns, the longitudinal return patterns corresponding to a longitudinal time of greater than one year; and
predict, based on the plurality of longitudinal return patterns and the longitudinal time, a future progression of a condition of the eye.

10. The system of claim 6, wherein the acoustic frequency is in a range between 48 and 120 megahertz (MHz).

11. The system of claim 6, wherein the one or more processors are configured to:
determine, based on the return pattern, that the transducer should be reoriented; and
provide, via a user interface, an indication that the transducer should be reoriented.

12. The system of claim 6, wherein the one or more processors are configured to:
compare the mechanical feature to a mechanical feature threshold; and
provide, via the user interface, an indication of the mechanical feature.

13. The system of claim 12, wherein the mechanical feature comprises a plurality of elasticities corresponding to a plurality of loci of the anatomical structure.

14. The system of claim 6, wherein the one or more processors are configured to:
present an indication of the mechanical features corresponding to the plurality of loci.

15. The system of claim 6, wherein the one or more processors are configured to:
predict a progression of a condition of the eye; and
provide, via the user interface, the prediction.

16. The system of claim 6, wherein the one or more processors are configured to:
during a first mode of operation, cause the transducer to receive a first portion of a return signal from ambient sources; and
during a second mode of operation temporally offset from the first mode of operation, cause the transducer to generate the ultrasonic pressure waves and receive a second portion of the return signal.

17. The system of claim 16, wherein the one or more processors are configured to:
generate a first portion of a data map from the first portion of the return signal, the first portion of the data map corresponding to a stationary position of the transducer relative to the eye; and
generate a second portion of a data map from the second portion of the return signal, the second portion of the data map corresponding to a moving position of the transducer relative to the eye.

18. A method of using an ultrasound probe to characterize a mechanical feature of an anatomical structure of an eye of a subject based on a return pattern indicative of the mechanical feature of the anatomical structure, the ultrasound probe comprising:
a transducer to generate ultrasonic pressure waves at an acoustic frequency to penetrate a surface of the eye to a depth of an anatomical structure of the eye;
a biocompatible end effector to contact the surface of the eye to propagate the ultrasonic pressure waves from the transducer to the eye; and
a sensor to detect the return pattern of the ultrasonic pressure waves.

19. The method of claim 18, wherein the characterization of the anatomical structure is predictive of a progression of myopia.

20. The method of claim 18, wherein the subject is human.
